(19) United States
(12) Patent Application Publication
(10) Pub. No.: US 2025/0228863 A1
(43) Pub. Date: Jul. 17, 2025

(54) Methods and Products to Screen and Treat Glioblastoma Multiforme and Other Cancers, Including Breast Cancers, Using A Combination of PI3KINASE Inhibitors With Checkpoint Inhibitors

(71) Applicant: GLOBAL CANCER TECHNOLOGY, INC., San Diego, CA (US)

(72) Inventors: Martha Karen Newell ROGERS, Kirkland, WA (US); Ekokobe FONKEM, Georgetown, TX (US); Richard TOBIN, Aurora, CO (US)

(21) Appl. No.: 18/854,796

(22) PCT Filed: Apr. 6, 2023
(86) PCT No.: PCT/US2023/065433

§ 371 Date: Oct. 7, 2024

Related U.S. Application Data
(60) Provisional application No. 63/329,096, filed on Apr. 8, 2022.
(60) Provisional application No. 63/368,499, filed on Jul. 15, 2022.

(51) Int. Cl. A61K 31/5377; A61K 31/553; A61K 31/585; A61K 39/00; A61K 45/06; A61P 35/00; C07K 16/28; G01N 33/50

(52) U.S. Cl. CPC A61K 31/5377; A61K 31/553; A61K 31/585; A61K 45/06; A61P 35/00; C07K 16/2818; C07K 16/2827; G01N 33/5017; A61K 2039/505

(57) ABSTRACT

The present invention recognizes that there is a long felt need for methods of treating cancer. A first aspect of the present invention generally relates to methods of screening for treatments of cancer. A second aspect of the present invention generally relates to compounds for treating cancer. A third aspect of the present invention generally relates to pharmaceutical compositions for treating cancer. A fourth aspect of the present invention generally relates to methods of treating cancer. A fifth aspect of the present invention generally relates to P13K inhibitors with checkpoint inhibitors for treating cancer. A sixth aspect of the present invention generally related to methods of treating cancers using P13K inhibitors with checkpoint inhibitors, or other immunotherapies. A seventh aspect of the present invention generally relates to methods of treating glioblastoma, breast cancer, and/or triple negative breast cancer using P13K inhibitors with checkpoint inhibitors, or other immunotherapies.

### PRIORITY STATEMENT

The present application:
claims benefit of priority to U.S. Provisional Application No. 63/329,096, filed Apr. 8, 2022, now expired; and
claims benefit of priority to U.S. Provisional Application No. 63/368,499, filed Jul. 15, 2022, now pending;
each of which is incorporated by reference in its entirety herein.

### TECHNICAL FIELD

The present invention relates generally to the field of treatment of cancer.

### BACKGROUND

Glioblastoma Multiforme (GBM) is a serious aggressive cancer of the central nervous system with no reported cure. Unlike breast and lung cancer, survival rates have not improved much over the past 40 years. GBM can progress from existing astrocytomas or develop de novo. About 90% of GBMs occur in patients, averaging 64 years as primary tumors, although it can occur at any age. Current approaches to treatment include resection, if possible, combined with chemotherapy, radiation, steroids and/or virus treatment. Despite treatments, the median survival is 9-22 months.

One of the most promising new strategies for the treatment of many cancers has been the development of therapies that target the body's own immune system, or its components, to fight the disease. Promising approaches in immunotherapy include drugs that target “immune checkpoint” molecules. For example, cancer-free survival rates for patients with advanced melanoma treated with immune checkpoint inhibitor (ICI) drugs are in the range of 30%-40%.

Although a positive outcome for some melanoma patients, the question remains why a large percentage of melanoma patients, roughly 60%-70%, do not respond favorably to otherwise blockbuster ICI drugs. Similarly, the question remains why ICI drugs are effective to some extent in treating certain forms of cancer, but why treatment of GBM patients with ICI drugs has not resulted in improved outcomes when compared with GBM patients treated with temozolomide (TMZ) or Avastin™, (currently used treatments for GBM).

The phosphatidylinositol 3-kinase (PI3K)/AKT/mTOR pathway is an important intracellular signaling pathway involved in numerous biological processes, including proliferation, apoptosis, angiogenesis, and glucose metabolism. The pathway is initiated by the binding of extracellular growth factors to receptor tyrosine kinases (RTKs) upon which PI3K is activated and subsequently activates the serine/threonine protein kinase B (PKB or AKT), leading to phosphorylation of a range of downstream substrates which mediate cellular responses. Hyper-activation of the (PI3K)/Akt pathway is frequently found in cancers, including glioblastoma and breast cancers, and the pathway plays a central role in the regulation of tumor cell survival, growth, angiogenesis, and metabolism. Numerous PI3K inhibitors including pan-PI3K inhibitors, isoform-selective and dual PI3K/mTOR inhibitors have exhibited favorable preclinical results and have entered clinical trials for a range of malignancies. Currently, there are a few PI3K inhibitors in phase I/II clinical trials for breast cancer.

The central issue that has limited the use of PI3K inhibitors for breast cancer, glioblastoma multiforme, and diffuse intrinsic pontine glioma (DIPG) is the potential toxicities of systemic delivery of these drugs throughout the body. One potentially advantageous therapeutic solution would be to create locally high drug concentrations only at the sites of tumor tissue, thereby sparing normal tissues from toxicity. Many cancer researchers have proposed the concept of pro-drugs and have placed an emphasis on potential activating agents such as tumor specific enzymes, light, or heat, however multiple issues arise. For example, enzymes are often heterogeneously distributed, inaccessible, and are often not entirely tumor specific. Heat and light are difficult to precisely apply deep within the tissue structure and blood flow can scatter and dissipate energy deposition.

Recent studies of tumor cell expression of Major Histocompatibility Complex class II (MHCII) molecules have identified at least, in part, a reason for the failure of ICI drugs to be effective in treating some melanoma patients and can also explain why ICI drugs have been ineffective to date in treating GBM. Specifically, recent studies have demonstrated that the tumors of patients that respond to ICI therapies are those whose tumors express cell surface MHC class II molecules. MHCII expression at the cell surface is well known to provide the first (and possibly required) signal for T-cell activation (a key cellular event in an anti-tumor immune response), and can, under appropriate conditions, cause cell death of the MHCII expressing cell. These MHCII-related studies have important implications for improving the potential efficacy of ICI drugs in treating various cancers and can generally support the hypothesis that MHC II expression on GBM cells, in combination with a blockade of immune check points, might provide a missing component that can be necessary to increase the frequency of patients that respond to ICI drugs via effective anti-tumor immunity. In the paper “Melanoma-specific MHC-II expression represents a tumor-autonomous phenotype and predicts response to anti-PD-1/PD-L1 therapy” the authors identified melanoma cell lines that expressed MHC II or they were able to induce MHC II expression on. They found that the expression of HLA-DR correlated with positive responses to PD-1-targeted immune checkpoint therapy in patients. They also found a positive correlation with HLA-DR expression and the percent of CD4+ T cell infiltration. While the authors hypothesize CD4+ helper cells are involved in this process, they did not identify the subsets of CD4+ cells, including the involvement of undesirable T regs that suppress an anti-tumor T cell response.

MHC II-encoded molecules are normally found on professional antigen presenting cells (APCs), including macrophages, dendritic cells, and B cells, but many cells can be induced to express the molecules and could potentially act as APCs. In humans, the MHC Class II molecule is known as the human leukocyte antigen gene complex (HLA Dr). MHC II molecules presents antigen (Ag) in its groove to the T cell receptor (TCR) expressed on T lymphocytes. T cell activation also requires a second co-signaling molecule, provided by interactions between the presenting cell and the T cell. With some co-stimulatory signals, the T cell becomes activated. With others, the CD4+ T cells that expand are regulatory and appear to suppress conventional T cell activation, most likely via the interaction between PD1 and PDL-1.

### SUMMARY

The present invention recognizes that there is a long felt need for methods of treating cancer.

A first aspect of the present invention generally relates to methods of screening for treatments of cancer.

A second aspect of the present invention generally relates to compounds for treating cancer.

A third aspect of the present invention generally relates to pharmaceutical compositions for treating cancer.

A fourth aspect of the present invention generally relates to methods of treating cancer.

A fifth aspect of the present invention generally relates to pharmaceutical compositions including PI3K inhibitors with checkpoint inhibitors, or other immunotherapies.

A sixth aspect of the present invention generally related to methods of treating cancers using PI3K inhibitors with checkpoint inhibitors, or other immunotherapies.

A seventh aspect of the present invention generally relates to methods of treating glioblastoma, breast cancer, and/or triple negative breast cancer using PI3K inhibitors with checkpoint inhibitors, or other immunotherapies.

### BRIEF DESCRIPTION OF THE FIGURES

FIG. 1. Gl261 mouse GBM cells were cultured for 24, 48 or 72 hours in the presence GCT.GLIO.1 at the indicated concentrations. Cells were then harvested and counted.

FIG. 2. GL261 cells were cultured with and without GCT.GLIO.1, for 72 hours, harvested, and stained with anti-PD-L1 as shown. The histograms indicate an increase in the cell surface expression of PD-L1 corresponding to an increased dose of GCT.GLIO.1 and indicate that increased PD-L1 can provide a target for immune checkpoint inhibitor therapy.

FIG. 3. GL261 cells were cultured with and without GCT.GLIO.1, at las indicated, for 7 days, harvested, and stained as indicated to determine changes in the cell surface expression of PD-L1 and MHC class II; or for the expression of a marker of autophagy, LC3. The increases in MHC class II and PD-L1 suggest that the drug would increase the likelihood of recognition by CD4 T cells and that increased PD-L1 would provide a target for immune checkpoint inhibitor therapy.

FIG. 4A and FIG. 4B. Effects of GCT.GLIO.1 on radiation sensitivity and Cell Cycle Arrest of GL261. In the next experiment, we sought to identify the optimum dose of radiation following treatment with GCT.Glio.1. In these studies, we treated with GCT.Glio.1 for 24 hours followed by increasing doses of irradiation, including 2, 4, and 8 Gy. The results show that retreatment with GCT.Glio.1 significantly amplified the response to radiation and caused significant increase in cells arrested at the G2 cell cycle checkpoint, see FIG. 5 through FIG. 6.

FIG. 5A and FIG. 5B. Human GBM cell line U251 treated with GCT.Glio.1, harvested, counted, and stained for changes in cell surface PD-L1, left panel, and MHC class II, right panel. The results show that GCT.Glio.1 induced growth arrest and increased cell surface PD-L1 and MHC class II (denoted as Mean Fluorescence Intensity (MFI)), potentially making these cells a better target for immunotherapy.

FIG. 6A and FIG. 6B. Human U251 cell lines were treated with GCT.Glio.1, harvested, counted, and assessed for changes in cell number and in dead cells. The results show that GCT.Glio.1 induced growth arrest, increasing as a function of increased doses of compound, left panel, and increased numbers of dead cells, right panel.

FIG. 7. Cell viability of human MCF7 breast cancer cells. Human MCF7 cells were treated with 1 uM Apelisib (Alp), 1 uM GCT.BC.1 (GCT), or DMSO control for 48 hours. Viability was determined by cleaved caspase-7 and was measured using a Caspase Glo assay (Promega) as described by the manufacturer.

FIG. 8. Cell viability of mouse TC11 breast cancer cells. TC11 cells were treated with 100 nM Paclitaxel (Pac), 100 nM Paclitaxel+1 uM Alpelisib (Alp), 0.50 uM GCT.BC.1 (GCT), 0.50 uM GCT.Glio.1+100 nM Paclitaxel, 0.50 uM GCT.BC.1+7.5 nM Fulvestrant, or DMSO control as indicated for 48 hours. Viability was determined by cleaved caspase-7 and was measured using a Caspase Glo assay (Promega) as described by the manufacturer.

FIG. 9. Proliferation of 66CL4 mouse breast cancer cells. 66CL4 mouse breast cancer cells were treated with the designated doses of GCT.BC.01. Proliferation was determined using the Agilent Biotek Biospa system and Biospa software.

FIG. 10. Luciferase flagged GL261 tumor cells were implanted into the caudate nucleus of C57B16 mice at day 0. Treatments began on day 9. Treatments included no treatment, GCT alone orally administered, anti-PD-1 plus DMSO vehicle, or GCT orally administered followed 24 hours later with the checkpoint inhibitor anti-PD1 as indicated. Tumor growth was confirmed by MRI. Kaplan Meier Survival Curve. The black line indicates animal survival for animals implanted with GL261; the red line indicates survival of animals treated with GCT.1; The blue line indicates animals treated with GCT.1 followed 24 hours with treatment with anti-PD1, with the cycle repeating. As of day 28, 3 animals in the combination group are alive.

FIG. 11. Luciferase flagged GL261 tumor cells were implanted into the caudate nucleus of C57B16 mice at day 0. Treatments began on day 9. Treatments included no treatment, GCT alone orally administered, anti-PD-1 plus DMSO vehicle, or GCT orally administered followed 24 hours later with the checkpoint inhibitor anti-PD1 as indicated. The GL261 tumors were measured using bioluminescence and monitored daily. In the upper panel, bioluminescence quantification indicates a significant delay in tumor growth when animals were treated with GCT.1 followed by the checkpoint inhibitor anti-PD-1.

FIG. 12A, FIG. 12B, and FIG. 12C. Structures for various compounds referred to in the Examples. (R)-NPT520-232 refers to GCT.2 (GCT.Glio.2), (−)-NPT520-337 refers to GCT.1 (GCT.Glio.1), and (−)-NPT520-338 refers to GCT.Glio.3.

FIG. 13A, FIG. 13B, and FIG. 13C. SEMA7A over-expressing (OE) cells respond poorly to endocrine therapy but become sensitive with the addition of PI3K inhibition. MCF7EV and SEMA7A OE cells were treated for 48 hours with FIG. 13A) fulvestrant (7.5 nM), FIG. 13B) Alpelisib (1 uM), or FIG. 13C) the combination.

FIG. 14A, FIG. 14B, FIG. 14C, FIG. 14D, and FIG. 14E. SEMA7A-expressing cells are sensitive to both Alpelisib and a novel PI3K inhibitor (GCT.BR.1) FIG. 14A) MCF7 cells treated with vehicle, 1 uM Alpelisib and 1 uM GCT.BR.1. Scalebar=10 microns. FIG. 14B) Quantification of A. FIG. 14C) MCF7 S7 OE spheroids in suspension were treated with vehicle (DMSO), 1 uM Alpelisib or 0.5 uM GCT and stained with Calcein green viability dye after 48 h. FIG. 14D) Tumor volume in MCF7 SEMA7 A OE-bearing NCG mice, treated with fulvestrant starting on day 30, every 5 days (arrows). FIG. 14E) size matched graphs.

FIG. 15. SK-BR-3 is a human breast cancer cell line that overexpresses the Her2 (Neu/ErbB-2) gene product. These cells display an epithelial morphology in tissue culture and are capable of forming poorly differentiated tumors in immunocompromised mice. MDA-MB-231 is a highly aggressive, invasive and poorly differentiated triple-negative breast cancer (TNBC) cell line as it lacks estrogen receptor (ER) and progesterone receptor (PR) expression, as well as HER2 (human epidermal growth factor receptor 2) amplification.

FIG. 16. SKBR3 cells treated with the labeled doses of GCT.BR.1. Histogram depicting the changes in expression of HER2 analyzed by flow cytometry. Treatment with GCT007 caused a dose-dependent increase in expression of HER 2 on triple negative SKRB3 breast cancer cells.

FIG. 17. 231 cells treated with the labeled doses of GCT.BR.1 (GCT007). Histogram depicting the changes in expression of HER2 analyzed by flow cytometry. Treatment with GCT007 caused a dose-dependent increase in expression of HER 2 on breast cancer 231 cells

FIG. 18. Quantification of the median fluorescence intensity of HER-2 expression on 231 cells.

### DETAILED DESCRIPTION OF THE INVENTION

### Definitions

Unless defined otherwise, all technical and scientific terms used herein have the same meaning as commonly understood by one of ordinary skill in the art to which this invention belongs. Generally, the nomenclature used herein and the laboratory procedures in cell culture, chemistry, microbiology, molecular biology, cell science, flow cytometry, and cell culture described below are well known and commonly employed in the art. Conventional methods are used for these procedures, such as those provided in the art and various general references such as are routinely known in the art. Where a term is provided in the singular, the inventors also contemplate the plural of that term. The nomenclature used herein and the laboratory procedures described below are those well-known and commonly employed in the art. As employed throughout the disclosure, the following terms, unless otherwise indicated, shall be understood to have the following meanings:

“Directly” refers to direct causation of a process that does not require intermediate steps.

“Indirectly” refers to indirect causation that requires intermediate steps.

A “subject” refers to a human or vertebrate mammal including but not limited to a dog, cat, horse, goat, and primate, for example, monkey. Thus, the present invention can also be used to treat cancer in non-human subjects. Preferably the subject is a human.

“Treat,” “treated,” or “treating” when used with respect to a disorder refers to a prophylactic treatment which increases the resistance of a subject to development of the disease or, in other words, decreases the likelihood that the subject will develop the disease as well as a treatment after the subject has developed the disease in order to fight the disease, prevent the disease from becoming worse, or slow the progression of the disease compared to in the absence of the therapy.

Other technical terms used herein have their ordinary meaning in the art that they are used, as exemplified by a variety of technical dictionaries.

### INTRODUCTION

The present invention recognizes that there is a long felt need for methods of treating cancer.

As a non-limiting introduction to the breath of the present invention, the present invention includes several general and useful aspects, including:
1) methods of screening for treatments of cancer;
2) compounds for treating cancer;
3) pharmaceutical compositions for treating cancer;
4) methods of treating cancer;
5) pharmaceutical compositions including PI3K inhibitors with checkpoint inhibitors, or other immune therapies;
6) methods of treating cancers using PI3K inhibitors with checkpoint inhibitors, or other immunotherapies; and
7) methods of treating glioblastoma, breast cancer, and/or triple negative breast cancer using PI3K inhibitors with checkpoint inhibitors, or other immunotherapies.

These aspects of the invention, as well as others described herein, can be achieved by using the methods, articles of manufacture and compositions of matter described herein. To gain a full appreciation of the scope of the present invention, it will be further recognized that various aspects of the present invention can be combined to make desirable embodiments of the invention.

### I Methods of Screening for Treatments of Cancer

The present invention includes methods of screening for treatments of cancer.

A first aspect of the present invention includes a method of screening at least one test compound, at least one procedure, or combinations thereof for their ability to make at least one cancer cell more susceptible to one or more treatments for the reduction of cancer cell numbers in a population of cells, including:
a. providing one or more cancer cells that expresses one or more markers;
b. providing one or more of said at least one test compound, at least one procedure, or combinations thereof;
c. contacting said one or more cancer cells with said one or more test compounds;
d. measuring the change in expression of said one or more markers by said one or more cancer cells in the presence or absence of said one or more test compounds;
   wherein a change in expression of said one or more markers in said one or more cancer cells in the presence of said one or more test compounds relative to in the absence of said one or more test compounds is reasonably predictive of said one or more cancer cells being more susceptible to said one or more treatments for the reduction of cancer cell numbers.

The activity of the compounds and combinations thereof used in accordance with the present invention can be determined by any method known in the art or shown in the examples. In one aspect of the present invention, the activity of an inhibitor or antagonist is determined by using various experimental animal models, including but not limited to, cancer animal models such as immune competent wild-type animals implanted with genetically compatible tumors; the SCID mouse model or nude mice with human tumor grafts.

Various in vitro and in vivo assays that test the activities of an inhibitor or antagonist are used in purification processes of an inhibitor or antagonist. The protocols and compositions of the invention are also preferably tested in vitro, and then in vivo, for the desired therapeutic or prophylactic activity, prior to use in humans.

Compounds for use in therapy can be tested in suitable and acceptable or accepted animal model systems prior to testing in humans, including but not limited to in rats, mice, chicken, cows, monkeys, rabbits, etc.

Toxicity and efficacy of the prophylactic and/or therapeutic protocols of the present invention can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and that therapeutic index can be expressed as the ratio LD₅₀/ED₅₀. Prophylactic and/or therapeutic agents that exhibit large therapeutic indices are preferred. While prophylactic and/or therapeutic agents that exhibit toxic side effects can be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage can vary within this range depending upon the dosage form employed and the route of administration utilized. For any agent used in the method of the invention, the therapeutically effective dose can be estimated initially from cell culture assays. A dose can be formulated in animal models to achieve a circulating plasma concentration range that includes the IC₅₀ (for example, the concentration of the test compound that achieves a half-maximal inhibition of symptoms) as determined in cell culture. Such information can be used to more accurately determine useful doses in humans. Levels in plasma can be measured, for example, by high performance liquid chromatography.

In certain aspects of the present invention, pharmaceutical compositions can include, for example, at least about 0.1% of an active compound. In other aspects of the present invention, the active compound can include between about 2% to about 75% of the weight of the unit, or between about 25% to about 60%, for example, and any range derivable therein.

The compounds and compositions described herein can be used alone or in conjugates with other molecules such as detection or cytotoxic agents in the detection and treatment methods of the invention, as described in more detail herein.

Typically, one of the components can include, or is coupled or conjugated to, a detectable label, for use in specific binding type assays as are known in the art. A detectable label is a moiety, the presence of which can be ascertained directly or indirectly. In some instances, detection of the label involves an emission of energy by the label. The label can be detected directly by its ability to emit and/or absorb photons or other atomic particles of a particular wavelength (for example, radioactivity, luminescence, optical or electron density, etc.). A label can be detected indirectly by its ability to bind, recruit and, in some cases, cleave another moiety which itself can emit or absorb light of a particular wavelength (for example, epitope tag such as the FLAG epitope, enzyme tag such as horseradish peroxidase, etc.). An example of indirect detection is the use of a first enzyme label which cleaves a substrate into visible products. The label can be of a chemical, peptide or nucleic acid molecule nature although it is not so limited. Other detectable labels include radioactive isotopes such as P³² or H³, luminescent markers such as fluorochromes, optical or electron density markers, etc., or epitope tags such as the FLAG epitope or the HA epitope, biotin, avidin, and enzyme tags such as horseradish peroxidase, β-galactosidase, etc. The label can be bound to a peptide during or following its synthesis. There are many different labels and methods of labeling known to those of ordinary skill in the art. Examples of the types of labels that can be used in the present invention include enzymes, radioisotopes, fluorescent compounds, colloidal metals, chemiluminescent compounds, and bioluminescent compounds. Those of ordinary skill in the art will know of other suitable labels for the peptides described herein, or will be able to ascertain such, using routine experimentation. Furthermore, the coupling or conjugation of these labels to the peptides of the invention can be performed using standard techniques common to those of ordinary skill in the art.

Another category of detectable labels includes but is not limited to diagnostic and imaging labels (generally referred to as in vivo detectable labels) such as for example magnetic resonance imaging (MRI): Gd(DOTA); for nuclear medicine: ²⁰¹Tl, gamma-emitting radionuclide 99mTc; for positron-emission tomography (PET): positron-emitting isotopes, (18)F-fluorodeoxyglucose ((18)FDG), (18)F-fluoride, copper-64, gadodiamide, and radioisotopes of Pb(II) such as 203Pb; 111 In.

The conjugations or modifications described herein employ routine chemistry, which chemistry does not form a part of the invention and which chemistry is well known to those skilled in the art of chemistry. The use of protecting groups and known linkers such as mono- and hetero-bifunctional linkers are well documented in the literature and will not be repeated here.

As used herein, “conjugated” means two entities stably bound to one another by any physiochemical means. It is important that the nature of the attachment is such that it does not impair substantially the effectiveness of either entity. Keeping these parameters in mind, any covalent or non-covalent linkage known to those of ordinary skill in the art can be employed. In some aspects of the present invention, covalent linkage is preferred. Noncovalent conjugation includes hydrophobic interactions, ionic interactions, high affinity interactions such as biotin-avidin and biotin-streptavidin complexation and other affinity interactions. Such means and methods of attachment are well known to those of ordinary skill in the art.

A variety of methods can be used to detect the label, depending on the nature of the label and other assay components. For example, the label can be detected while bound to the solid substrate or subsequent to separation from the solid substrate. Labels can be directly detected through optical or electron density, radioactive emissions, nonradiative energy transfers, etc. or indirectly detected with antibody conjugates, streptavidin-biotin conjugates, etc. Methods for detecting the labels are known in the art.

### A. Test Compounds, Procedures, or Combinations Thereof

An aspect of the present invention includes wherein said test compounds, procedures, or combinations thereof include at least one chemical compound, at least one chemotherapy, at least one immunotherapy, at least one radiation, at least one low temperature, at least one high temperature, at least one laser, at least one surgery, at least one nanoparticle delivery system, or a combination thereof.

Another aspect of the present invention includes wherein said nanoparticle delivery system includes at least one scintillator.

A further aspect of the present invention includes wherein said nanoparticle delivery system includes at least one cleavable linker.

An additional aspect of the present invention includes wherein said cleavable linker is between the drug and the nanoparticle.

### B. Cancer Cells

An aspect of the present invention includes wherein said cancer cells are derived from endoderm, mesoderm, ectoderm, or a combination thereof.

Another aspect of the present invention includes wherein said cancer cells include carcinoma cells, sarcoma cells, leukemia cells, lymphoma cells, myeloma cells, or a combination thereof.

A further aspect of the present invention includes wherein said cancer cells include skin cells, melanoma cells, non-melanoma cells, brain cells, prostate cells, breast cells, colorectal cells, kidney cells, bladder cells, non-Hodgkin's lymphoma cells, thyroid cells, endometrial cells, head cells, neck cells, brain cells, glioblastoma cells, or a combination thereof.

### C. More Susceptible to Treatments

An additional aspect of the present invention includes wherein said more susceptible to treatments includes said cancer cells being more susceptible to cell death, reduction in growth rate, increase in doubling time, more susceptible to DNA damage, less able to repair DNA damage, more likely to be recognized by an immune cell, or a combination thereof.

### D. Treatments for Reduction of Cancer Cell Numbers

An aspect of the present invention includes wherein said treatments for reduction of cancer cell numbers includes at least one chemotherapy, at least one immunotherapy, at least one radiation, at least one low temperature, at least one high temperature, at least one laser, at least one surgery, at least one nanoparticle delivery system, or a combination thereof.

### E. Population of Cells

Another aspect of the present invention includes wherein said populations of cells includes a pure culture, a mixed culture, a biopsy, in vivo, in situ, or a combination thereof.

### F. Markers

A further aspect of the present invention includes wherein said one or more markers include one or more of:
i. PD-L1;
ii. MHC Class II;
iii. CD80;
iv. CD86;
v. CD40;
vi. CD95;
vii. MHC Class I
viii. Lag-3
ix. Vista
x. LC3;
xi. one or more of: CD95, CD44, CD47, TRA-1-60, SSEA-1, EpCam, ALDH1A1, Lgr5, CD13, CD19, CD20, CD24, CD26, CD27, CD34, CD38, CD44, CD45, CD47, CD49f, CD66c, CD90, CD166, TNFRSF16, CD105, CD133, CD117/c-kit, CD138, CD151, CD166, and combinations thereof; and
xii. combinations thereof.

### G. Measuring Changes in Expression

An additional aspect of the present invention includes wherein said measuring change in expression includes immunological methods, nucleic acid methods, proteomic methods, flow cytometric methods, genomic methods, or a combination thereof a pure culture, a mixed culture, a biopsy, or a combination thereof.

### H. Changes in Expression

An aspect of the present invention includes wherein said change in expression includes an increase, decrease, or combination thereof from a pure culture, a mixed culture, a biopsy, or a combination thereof.

### I. Reasonably Predictive

Another aspect of the present invention includes wherein said reasonably predictive includes being a statistically significant difference at a p value of less than 0.10, 0.05, or as acceptable to those in the art.

### J. Further Testing

A further aspect of the present invention includes further comprising testing said cancer cells with said one or more test compounds with one or more treatments for the reduction of cancer cell numbers.

### II Compounds for Treating Cancer

The present invention also includes compounds for treating cancer.

A second aspect of the present invention includes a compound identified by the method of aspect I of the present invention.

### III Pharmaceutical Compositions for Treating Cancer

The present invention includes pharmaceutical compositions for treating cancer.

A third aspect of the present invention includes a pharmaceutical composition, comprising: a compound of the present invention in a pharmaceutically effective amount in a pharmaceutically acceptable carrier.

An aspect of the present invention includes a pharmaceutical composition of the present invention further comprising at least one additional cancer treatment.

Another aspect of the present invention includes a pharmaceutical composition of the present invention wherein said treatment includes at least one chemotherapy, at least one immunotherapy, at least one radiation, at least one low temperature, at least one high temperature, at least one laser, at least one surgery, at least one nanoparticle delivery system, or a combination thereof.

A further aspect of the present invention includes a pharmaceutical composition, comprising:
at least one pan PI3K inhibitor;

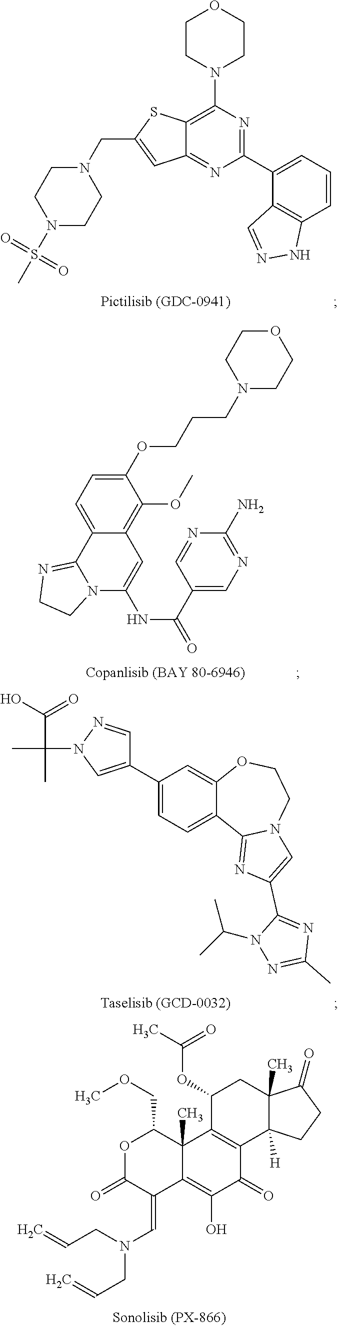

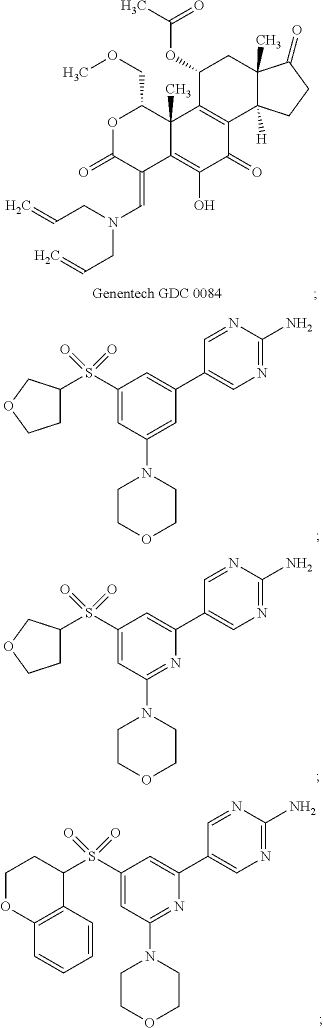 [SMILES: C=CCN(/C=C1\C(=O)O[C@H](COC)[C@@]2(C)C1=C(O)C(=O)C1=C2[C@H](OC(C)=O)C[C@]2(C)C(=O)CC[C@@H]12)CC=C ; Nc1ncc(-c2cc(N3CCOCC3)cc(S(=O)(=O)C3CCOC3)c2)cn1 ; Nc1ncc(-c2cc(S(=O)(=O)C3CCOC3)cc(N3CCOCC3)n2)cn1 ; Nc1ncc(-c2cc(S(=O)(=O)C3CCOc4ccccc43)cc(N3CCOCC3)n2)cn1]

or a combination thereof.

### IV Methods of Treating Cancer

The present invention also includes methods of treating cancer.

A fourth aspect of the present invention includes a method of treating a subject with cancer, comprising:
a. providing a subject in need of treatment for at least one cancer;
b. providing at least one compound or pharmaceutical composition of the present invention or a combination thereof,
c. administering a pharmaceutically effective amount of said at least one compound to said subject;
wherein said subject is treated for said at least one cancer.

An aspect of the present invention further comprising administering at least one treatment to said subject.

Another aspect of the present invention includes wherein said treatment includes at least one chemotherapy, at least one immunotherapy, at least one radiation, at least one low temperature, at least one high temperature, at least one laser, at least one surgery, at least one nanoparticle delivery system, or a combination thereof.

V PI3K Inhibitors with Checkpoint Inhibitors or Other Immune Therapies for Treating Cancer-Pharmaceutical Compositions

The present invention includes PI3K inhibitors with checkpoint inhibitors or other immune therapies for treating cancer. The present invention can include other components as well as described herein, such as but not limited to CAR-T cells and antibodies.

A fifth aspect of the present invention includes a pharmaceutical composition, comprising:
a) at least one PI3K inhibitor in a pharmaceutically effective amount in a pharmaceutically acceptable carrier; and
b) at least one checkpoint inhibitor or other immune therapy, including CAR T cells or antibodies, in a pharmaceutically effective amount.

An aspect of the present invention includes wherein said at least one PI3K inhibitor and said checkpoint inhibitor are provided together or separately.

### A. PI3K Inhibitors and Agonists

In some aspects of the present invention PI3K inhibitors can also be administered alone or in combination with other components of the present invention.

Any PI3K Inhibitor can be used in the present invention. Many are disclosed herein, and additional PI3K inhibitors are disclosed in U.S. Published Patent Application No. 2021/0163462 A1 to Stocking et al., published Jun. 3, 2021; U.S. Pat. No. 11,492,348 to Stocking et al., issued Nov. 8, 2022; WO 2009/066087 to Hoffman-La Roche et al, published May 28, 2009; WO 2007/084786 to Novartis, published Jul. 26, 2007; U.S. Pat. No. 8,921,361 to Cmiljanovic et al, issued Dec. 30, 2014; each of which is incorporate by reference herein in its entirety.

Preferred PI3K inhibitors are disclosed in U.S. Pat. No. 11,492,348 to Stocking et al., issued Nov. 8, 2022, such as but not limited to the following:

In one aspect, the present disclosure provides a compound of Formula (I):

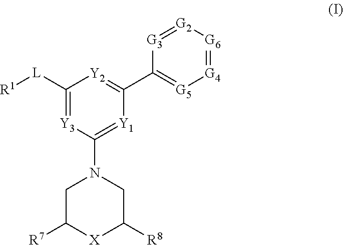

wherein
R¹ is —(CR^{a}R^{b})ₘ-aryl, —CH═CH-aryl, —(CR^{c}R^{d})ₙ-heteroaryl, —(CR^{e}R^{f})ₒ-heterocycloalkyl, or —(CR^{g}R^{h})ₚ-cycloalkyl; wherein
   m, n, o, and p are each independently 0, 1, or 2;
   R^{a}, R^{b}, R^{c}, R^{d}, R^{e}, R^{f}, R^{g}, and R^{h} are each independently H, halo, or C₁₋₄alkyl,
   or R^{a} and R^{b} are taken together with the carbon to which they are attached to form a cycloalkyl ring,
   or R^{a} and R^{b} are taken together to form ═CH₂ or ═O;
   each aryl, heteroaryl, heterocycloalkyl, or cycloalkyl present in R¹ is unsubstituted or substituted with one or two R^{x} substituents;
      wherein each R^{x} substituent is independently halo, C₁₋₄alkyl, cycloalkyl, —C₁₋₂-haloalkyl, —OH, —OC₁₋₄alkyl, —O—C₁₋₂-haloalkyl, cyano, —C(O) C₁₋₄alkyl, —C(O)NRⁱR^{j}), —SO₂C₁₋₄alkyl, —SO₂NR^{k}R^{l}, —NR^{q}R^{r}, —C(O)-cycloalkyl, —C(O)-aryl (optionally substituted with methyl or halo), —CO₂C₁₋₄alkyl, —CO₂aryl, —C(O) CH₂-aryl (optionally substituted with methyl or halo), —CH₂-aryl (optionally substituted with methyl or halo), or monocyclic heterocycloalkyl (optionally substituted with methyl, —C(O) C₁₋₄alkyl, or —CO₂C₁₋₄alkyl);
         wherein Rⁱ, R^{j}, R^{k}, and R^{l} are each independently H, C₁₋₄alkyl, —C₁₋₄alkyl-OH, or —C₁₋₄alkyl-O—C₁₋₄alkyl,
         wherein R^{q} and R^{r} are each independently H, C₁₋₄alkyl, —C₁₋₄alkyl-OH, —C₁₋₄alkyl-O—C₁₋₄alkyl, —C(O) C₁₋₄alkyl, —CO₂C₁₋₄alkyl, or —SO₂C₁₋₄alkyl;
L is absent, —S(O)₂—, —C(O)—, —O—, —CH₂—, —CF₂—, C(CH₃)₂, —C(═CH₂)—, or —CR^{s}R^{t}—; wherein R^{s} and R^{t} are independently H or alkyl, or R^{s} and R^{t} are taken together with the carbon atom to which they are attached to form a cycloalkyl ring;
X is O, S, NH, N(CO₂C₁₋₄alkyl), N(SO₂C₁₋₄alkyl), N(SO₂cycloalkyl), or CH₂;
Y₁, Y₂, and Y₃ are each independently CH or N; wherein when L is other than —S(O)₂—, Y₂ and Y₃ are each CH;
G₂ is N or CR²;
G₃ is N or CR³;
G₄ is N, NR^{4b}, or CR^{4a};
G₅ is N or CR⁵, and
G₆ is N or CR⁶;
   wherein R², R³, R^{4a}, R⁵, and R⁶ are each independently hydrogen, halogen, —OH, -alkyl, —Oalkyl, -haloalkyl, —O-haloalkyl, or —NR^{u}R^{v};
   or R^{4b} is taken together with R⁶ and the atoms to which they are attached to form a heteroaryl or heterocyclic ring; wherein the heteroaryl ring comprising R^{4b} and R⁶ comprises no more than one N and is optionally substituted with alkyl, and the heterocyclic ring comprising R^{4b} and R⁶ is optionally substituted with oxo,
   R^{u} is H or C₁₋₄alkyl;
   R^{v} is H, C₁₋₄alkyl, monocyclic cycloalkyl, —C(O) C₁₋₄alkyl, or —C(O)NR^{w}R^{y};
      wherein each alkyl present in R^{v} is unsubstituted or substituted with —OH, —NH₂, —NH(C₁₋₄alkyl), or —N(C₁₋₄alkyl)₂,
      R^{w} and R^{y} are each independently H or C₁₋₄alkyl;
      wherein

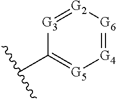

is not unsubstituted phenyl; and
R⁷ and R⁸ are each independently hydrogen or C₁₋₄alkyl,
or R⁷ and R⁸ are taken together to form —CH₂CH₂—;
or a pharmaceutically acceptable salt thereof.

In certain embodiments, the compound of Formula (I) is a compound selected from those species described or exemplified in the detailed description herein.

In some embodiments of Formula (I) or any variation thereof, R¹ is —(CR^{a}R^{b})ₘ-aryl. In some embodiments, R¹ is (CR^{c}R^{d})ₙ-heteroaryl. In some embodiments, R¹ is (CR^{e}R^{f})ₒ-heterocycloalkyl or (CR^{g}R^{h})ₚ-cycloalkyl.

In some embodiments of Formula (I) or any variation thereof, L is —S(O)₂—. In some embodiments, L is —C(O)—, —CH₂—, —CF₂—, C(CH₃)₂, —C(═CH₂)—, or —CR^{s}R^{t}—. In other embodiments, L is —C(O)—, —O—, —CH₂—, —CF₂—, C(CH₃)₂, —C(═CH₂)—, or —CR^{s}R^{t}—. In some embodiments, L is absent.

Provided in other aspects are compounds of Formula (II):

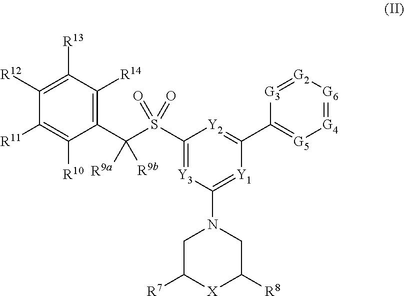

wherein
X is O, S, NH, N(CO₂C₁₋₄alkyl), N(SO₂C₁₋₄alkyl), N(SO₂cycloalkyl), or CH₂;
Y₁, Y₂, and Y₃ are each independently CH or N;
G₂ is N or CR²;
G₃ is N or CR³;
G₄ is N, NR^{4b}, or CR^{4a};
G₅ is N or CR⁵; and
G₆ is N or CR⁶;
   wherein R², R³, R^{4a}, R⁵, and R⁶ are each independently hydrogen, halogen, —OH, -alkyl, —Oalkyl, -haloalkyl, —O-haloalkyl, or —NR^{u}R^{v};
   or R^{4b} is taken together with R⁵ and the atoms to which they are attached to form a heteroaryl or heterocyclic ring; wherein the heteroaryl ring comprising R^{4b} and R⁶ comprises no more than one N and is optionally substituted with alkyl, and the heterocyclic ring comprising R^{4b} and R⁶ is optionally substituted with oxo,
   R^{u} is H or C₁₋₄alkyl;
   R^{v} is H, C₁₋₄alkyl, monocyclic cycloalkyl, —C(O) C₁₋₄alkyl, or —C(O)NR^{w}R^{y};
      wherein each alkyl present in R^{v} is unsubstituted or substituted with —OH, —NH₂, —NH(C₁₋₄alkyl), or —N(C₁₋₄alkyl)₂, and
      R^{w} and R^{y} are each independently H or C₁₋₄alkyl;
      wherein

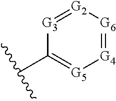

is not unsubstituted phenyl;
R⁷ and R⁸ are each independently hydrogen or C₁₋₄alkyl,
or R⁷ and R⁸ are taken together to form —CH₂CH₂—;
R^{9a} and R^{9b} are each independently hydrogen or halogen;
R¹⁰, R¹¹, R¹², R¹³, and R¹⁴ are each independently hydrogen, halogen, —OH, —CN, -alkyl, —Oalkyl, -haloalkyl, heterocycloalkyl, —O-haloalkyl, —SO₂C₁₋₄alkyl, or —NR^{aa}R^{bb};
   R^{aa} is hydrogen, C₁₋₄alkyl, or —C₁₋₄alkyl-OH;
   R^{bb} is hydrogen or C₁₋₄alkyl;
or R^{9a} is taken together with R¹⁰ and the interposed atoms to form a heteroaryl or heterocyclic ring;
or R¹¹ is taken together with R¹² and the atoms to which they are attached to form a heteroaryl or heterocyclic ring;
or a pharmaceutically acceptable salt thereof.

Provided in other aspects are compounds of Formula (III):

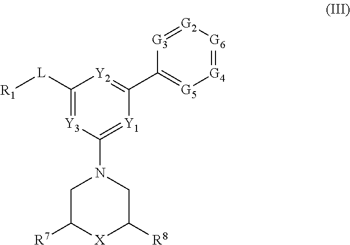

wherein
R¹ is —(CR^{a}R^{b})ₘ-aryl, —CH═CH-aryl, (CR^{c}R^{d})ₙ-heteroaryl, (CR^{c}R^{f})ₒ-heterocycloalkyl, or (CR^{g}R^{h})ₚ-cycloalkyl, wherein when L is SO₂, the heteroaryl and the heterocycloalkyl present in R¹ are each monocyclic;
   m is 0 or 2;
   n, o, and p are each independently 0, 1, or 2;
   R^{a}, R^{b}, R^{c}, R^{d}, R^{e}, R^{f}, R^{g}, and R^{h} are each independently H, halo, or C₁₋₄alkyl,
   or R^{a} and R^{b} are taken together with the carbon to which they are attached to form a cycloalkyl ring,
   or R^{a} and R^{b} are taken together to form ═CH₂ or ═O;
   each aryl, heteroaryl, heterocycloalkyl, or cycloalkyl present in R¹ is unsubstituted or substituted with one or two R substituents;
   wherein each R^{x} substituent is independently halo, C₁₋₄alkyl, cycloalkyl, —C₁₋₂-haloalkyl, —OH, —OC₁₋₄alkyl, —O—C₁₋₂-haloalkyl, cyano, —C(O) C₁₋₄alkyl, —C(O)NRⁱR^{j}, —SO₂C₁₋₄alkyl, —SO₂NR^{k}R^{l}, —NR^{q}R^{r}, —C(O)-cycloalkyl, —C(O)-aryl (optionally substituted with methyl or halo), —CO₂C₁₋₄alkyl, —CO₂aryl, —C(O) CH₂-aryl (optionally substituted with methyl or halo), —CH₂-aryl (optionally substituted with methyl or halo), or monocyclic heterocycloalkyl (optionally substituted with methyl, —C(O) C₁₋₄alkyl, or —CO₂C₁₋₄alkyl);
   wherein Rⁱ, R^{j}, R^{k}, and R^{l} are each independently H, C₁₋₄alkyl, —C₁₋₄alkyl-OH, or —C₁₋₄alkyl-O—C₁₋₄alkyl,
   wherein R^{q} and R^{r} are each independently H, C₁₋₄alkyl, —C₁₋₄alkyl-OH, —C₁₋₄alkyl-O—C₁₋₄alkyl, —C(O) C₁₋₄alkyl, —CO₂C₁₋₄alkyl, or —SO₂C₁₋₄alkyl;
or R¹ is

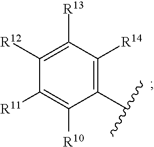

wherein
R¹⁰, R¹¹, R¹², R¹³, and R¹⁴ are each independently hydrogen, halogen, —OH, —CN, -alkyl, —Oalkyl, -haloalkyl, heterocycloalkyl, —O-haloalkyl, —SO₂C₁₋₄alkyl, or —NR^{aa}R^{bb},
R^{aa} is hydrogen, C₁₋₄alkyl, or —C₁₋₄alkyl-OH;
R^{bb} is hydrogen or C₁₋₄alkyl;
or R¹⁰ is taken together with R¹¹ and the atoms to which they are attached to form a heteroaryl or heterocyclic ring;
or R¹¹ is taken together with R¹² and the atoms to which they are attached to form a heteroaryl or heterocyclic ring;
L is absent, —S(O)₂—, —C(O)—, —O—, —CH₂—, —CF₂—, C(CH₃)₂, —C(═CH₂)—, or —CR^{s}R^{t}—; where R⁵ and R^{t} are independently H or alkyl, or R^{s} and R^{t} are taken together with the carbon atom to which they are attached to form a cycloalkyl ring;
X is O, S, NH, N(CO₂C₁₋₄alkyl), N(SO₂C₁₋₄alkyl), N(SO₂cyclo-alkyl), or CH₂;
Y₁, Y₂, and Y₃ are each independently CH or N; wherein when L is other than —S(O)₂—, Y₂ and Y₃ are each CH;
G₂ is N or CR²;
G₃ is N or CR³,
G₄ is N, NR^{4b}, or CR^{4a};
G₅ is N or CR⁵; and
G₆ is N or CR⁶;
   wherein R², R³, R^{4a}, R⁵, and R⁶ are each independently hydrogen, halogen, —OH, -alkyl, —Oalkyl, -haloalkyl, —O-haloalkyl, or —NR^{u}R^{v};
   or R^{4b} is taken together with R⁶ and the atoms to which they are attached to form a heteroaryl or heterocyclic ring; wherein the heteroaryl ring comprising R^{4b} and R⁶ comprises no more than one N and is optionally substituted with alkyl, and the heterocylic ring comprising R^{4b} and R⁶ is optionally substituted with oxo,
   R^{u} is H or C₁₋₄alkyl;
   R^{v} is H, C₁₋₄alkyl, monocyclic cycloalkyl, —C(O) C₁₋₄alkyl, or —C(O)NR^{w}R^{y};
wherein each alkyl present in R^{v} is unsubstituted or substituted with —OH, —NH₂, —NH(C₁₋₄alkyl), or —N(C₁₋₄alkyl)₂,
   R^{w} and R^{y} are independently H or C₁₋₄alkyl;
   wherein

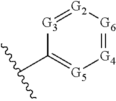

is not unsubstituted phenyl; and
R⁷ and R⁸ are each independently hydrogen or C₁₋₄alkyl,
or R⁷ and R⁸ are taken together to form —CH₂CH₂—;
or a pharmaceutically acceptable salt thereof.

In some embodiments of any of the compounds of Formula (I), (II), or (III), Y₁, Y₂, and Y₃ are each CH. In some embodiments, Y₁ is N and Y₂ and Y₃ are each CH. In some embodiments, Y₂ is N and Y₁ and Y₃ are each CH. In some embodiments, Y₃ is N and Y₁ and Y₂ are each CH.

In some embodiments of any of the compounds of Formula (I), (II), or (III), X is O. In some embodiments, X is NH, N(CO₂C₁₋₄alkyl), N(SO₂C₁₋₄alkyl), or N(SO₂cyclo-alkyl).

In some embodiments of any of the compounds of Formula (I), (II), or (III), G₂ and G₄ are each N, and G₆ is CR⁶. In some embodiments of any of the compounds of Formula (I), (II), or (III), G₃ is CR³ and G₅ is CR⁵. In some embodiments of any of the compounds of Formula (I), (II), or (III), one of G₂ and G₄ is N.

In some embodiments of any of the compounds of Formula (I), (II), or (III), R⁶ is —NR^{u}R^{v}. In some embodiments of any of the compounds of Formula (I), (II), or (III), R^{4b} is taken together with R⁶ and the atoms to which they are attached to form a heteroaryl or heterocyclic ring.

In a further aspect, the present disclosure provides a pharmaceutical composition comprising at least one compound of Formula (I), (II), or (III) or a pharmaceutically acceptable salt thereof. Pharmaceutical compositions according to the embodiments may further comprise a pharmaceutically acceptable excipient. Provided in some embodiments is a pharmaceutical composition that contains (a) at least one compound of Formula (I), (II) or (III), or a pharmaceutically acceptable salt thereof, and (b) a pharmaceutically acceptable excipient.

The PI3K inhibitors can be administered in combination with other anti-cancer agents such as checkpoint inhibitors, CAR T cells, or antibodies. The PI3K inhibitors can be administered by any appropriate formulation, dose, route of administration, and regime. Although the PI3K inhibitors of the present invention are preferably administered locally, they can be administered by any appropriate route of administration. Any other agent can be administered by any appropriate route of administration, including systemically or locally.

PI3K antagonists as well as PI3K inhibitors can also be used alone or in combination with other components of the present invention. Any PI3K agonist can be used in the present invention. Many are disclosed herein, and additional PI3K agonists are disclosed in U.S. Published PCT Patent Application WO 2019/199865 and U.S. Pat. No. 11,492,348, each of which is incorporate by reference herein in its entirety.

### B. Checkpoint Inhibitors

In some aspects of the present invention Checkpoint Inhibitors can also be administered alone or in combination with other components of the present invention.

Any Checkpoint Inhibitor can be used in the present invention. Many are disclosed herein, and additional Checkpoint Inhibitors are disclosed in, for example, PMID: 27054314 and https://doi.org/10.3389/fimmu.2020.01508, each of which is incorporate by reference herein in its entirety.

Inhibitory checkpoint molecules include, but are not limited to: PD-1, PD-L1, PD-L2, TIM-3, VISTA, A2AR, B7-H3, B7-H4, B7-H6, BTLA, CTLA-4, IDO, KIR and LAG3. CTLA-4, PD-1, and ligands thereof are members of the CD28-B7 family of co-signaling molecules that play important roles throughout all stages of T-cell function and other cell functions. CTLA-4, Cytotoxic T-Lymphocyte-Associated protein 4 (CD152), is involved in controlling T cell proliferation.

The PD-1 receptor is expressed on the surface of activated T cells (and B cells) and, under normal circumstances, binds to its ligands (PD-L1 and PD-L2) that are expressed on the surface of antigen-presenting cells, such as dendritic cells or macrophages. This interaction sends a signal into the T cell and inhibits it. Cancer cells take advantage of this system by driving high levels of expression of PD-L1 on their surface. This allows cancer cells to gain control of the PD-1 pathway and switch off T cells expressing PD-1 that can enter the tumor microenvironment, thus suppressing the anticancer immune response. Pembrolizumab (formerly MK-3475 and lambrolizumab, trade name KEYTRUDA®) is a human antibody used in cancer immunotherapy and targets the PD-1 receptor.

Some aspects of the present invention further involve administering a checkpoint inhibitor to the subject. In some aspects of the present invention, the checkpoint inhibitor can be an antibody selected from an anti-CTLA4 antibody or antigen-binding fragment thereof that specifically binds CTLA4, an anti-PD1 antibody or antigen-binding fragment thereof that specifically binds PD1, an anti-PD-L1 antibody or antigen-binding fragment thereof that specifically binds PD-L1, an antibody selected from an anti-VISTA antibody or antigen-binding fragment thereof that specifically binds VISTA, or a combination thereof. In other aspects of the present invention the checkpoint inhibitor can be an anti-PD-L1 antibody selected from atezolizumab, avelumab, durvalumab, or a combination thereof. In other aspects of the present invention, the checkpoint inhibitor is an anti-CTLA-4 antibody can be selected from tremelimumab or ipilimumab, or a combination thereof. In yet other embodiments of the present invention the checkpoint inhibitor can be an anti-PD1 antibody can be selected from nivolumab or pembrolizumab, or a combination thereof.

The checkpoint inhibitor, in some aspects of the present invention, is a molecule such as a monoclonal antibody, a humanized antibody, a fully human antibody, a fusion protein, a small molecule, or a combination thereof. For example, the checkpoint inhibitor can inhibit a checkpoint protein which can be CTLA-4, PDL1, PDL2, PD1, B7-H3, B7-H4, B7-H6, BTLA, HVEM, TIM3, GAL9, LAG3, VISTA, KIR, 2B4, CD160, CGEN-15049, CHK 1, CHK2, A2aR, B-7 family ligands, or a combination thereof. Ligands of checkpoint proteins include but are not limited to CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands, or a combination thereof. In some aspects of the present invention the anti-PD-1 antibody can be BMS-936558 (nivolumab). In other aspects of the present invention the anti-CTLA-4 antibody can be ipilimumab (trade name Yervoy®, formerly known as MDX-010 and MDX-101). In another aspect of the present invention, the checkpoint inhibitor can be J43 (an anti-PD1 antibody), RMP1-14 (an anti-PD1 antibody), atezolizumab (TECENTRIQ®; an anti-PDL1 antibody), or a combination thereof. In yet other aspects of the present invention, the checkpoint inhibitor can be pembrolizumab.

Pembrolizumab is a potent humanized immunoglobulin G4 monoclonal antibody with high specificity of binding to the PD-1 receptor, thus inhibiting its interaction with PD-L1 and programmed cell death 1 ligand 2. Based on preclinical in vitro data, pembrolizumab has high affinity and potent receptor blocking activity for PD-1. Pembrolizumab has an acceptable preclinical safety profile and is in clinical development as an IV immunotherapy for advanced malignancies. KEYTRUDA® (pembrolizumab) is approved for the treatment of patients across a number of indications. Pembrolizumab is approved for use in several cancer types, and is under investigation in several phases of clinical development for many more.

Preferred checkpoint inhibitors include but are not limited to at least one anti-CTLA-4 inhibitor, at least one PD-1 inhibitor, at least one anti-PD-L1 inhibitor, or a combination thereof. The preferred anti-CTLA-4 inhibitor includes but is not limited to ipilimumab. The preferred PD-1 inhibitor includes but is not limited to pembrolizumab, nivolumab, cemiplimab (trade name LIBTAYO®), and dostarlimab (trade name JEMPERLI®), or a combination thereof. The preferred anti-PD-L1 inhibitor includes but is not limited to atezolizumab (trade name TECENTRIQ®), durvalumab (trade name IMFINZI®), and Avelumab (trade name BAVENCIO®), or a combination thereof.

In some aspects of the present invention, the present invention includes administering at least one immune checkpoint inhibitor, as described herein or as otherwise known. In some aspects of the present invention, combinations of immune checkpoint inhibitors are administered.

### C. CAR-T Cells

In some aspects of the present invention cellular therapy such as CAR-T cells can also be administered alone or in combination with other components of the present invention.

Any appropriate CAR-T cell can be used in the present invention. Many are disclosed herein, and additional CAR-T cells are known in the literature.

Preferably, the PI3K inhibitor can be administered with such CAR-T cells. A CAR-T cell, as used herein, refers to T cells into which a chimeric antigen receptor has been introduced to redirect the receptor specificity towards an antigen of choice. Such receptors include an ectodomain that recognizes antigen independent of MHC restriction, in combination with cytoplasmic signaling domains. Many different proteins can be introduced into the T cells as the ectodomain of the chimeric antigen receptors. Examples include, but are not limited to, a nanobody, a monoclonal antibody, a humanized antibody, a chimeric antibody, a human antibody, an antibody fragment, or a combination thereof.

The CAR-T cell can be a cell (for example, T cell) engineered to express a CAR wherein the CAR-T cell preferably exhibits an antitumor property. The CAR can be engineered to include an ectodomain peptide fused to an intracellular signaling domain of the T cell antigen receptor complex Zeta chain (for example, CD3 Zeta). The CAR when expressed in a T cell is able to redirect antigen recognition based on the antigen binding specificity. The antigen binding peptide is preferably fused with an intracellular domain from one or more of a costimulatory molecule and a Zeta chain. In some aspects of the present invention, the antigen binding peptide is fused with one or more intracellular domains selected from the group of a CD137 (4-1BB) signaling domain, a CD28 signaling domain, a CD3 Zeta signal domain, and a combination thereof.

In some aspects of the present invention the invention involves administering to a subject having cancer an antigen specific CAR-T cell, with or without an PI3Kinase inhibitor. The CAR-T cell can have, for example, a modified PI3Kinase receptor, in an effective amount to treat the cancer.

### D. Antibodies Against Cancer Cells

Another form of anti-cancer therapy involves administering an antibody specific for a cell surface antigen of, for example, a cancer cell.

In some aspects of the present invention antibodies against cancer cells can also be administered alone or in combination with other components of the present invention.

Any appropriate antibody against cancer cells can be used in the present invention. Many are disclosed herein, and additional antibodies against cancer cells are known in the art.

In one aspect of the present invention, the antibody can be Ributaxin, Herceptin, Rituximab, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, BABS, anti-FLK-2, MDX-260, ANA Ab, SMART 1D10 Ab, SMART ABL 364 Ab, ImmuRAIT-CEA, and combinations thereof. Other antibodies include but are not limited to anti-CD20 antibodies, anti-CD40 antibodies, anti-CD19 antibodies, anti-CD22 antibodies, anti-HLA-DR antibodies, anti-CD80 antibodies, anti-CD86 antibodies, anti-CD54 antibodies, anti-CD69 antibodies, and combinations thereof. These antibodies are available from commercial sources or can be synthesized de novo.

Other cancer therapies and their dosages, routes of administration and recommended usage are known in the art and have been described in such literature.

VI PI3K Inhibitors with Checkpoint Inhibitors or Other Immune Therapies Including Car-T Cells or Antibodies for Treating Cancer

The present invention also includes methods of treating cancer.

A sixth aspect of the present invention includes A method of treating a subject with cancer, including: a) providing a subject in need of treatment for at least one cancer; b) providing at least one compound or pharmaceutical composition of the present invention, or a combination thereof; and c) administering a pharmaceutically effective amount of the at least one compound to said subject; wherein the subject is treated for said at least one cancer.

In some aspects, the present invention provides methods for treating a cancer (including ameliorating one or more symptoms thereof) in a subject refractory to existing single agent therapies for such a cancer, said methods comprising administering to said subject a therapeutically effective amount of a composition comprising an inhibitor or antagonist and a therapeutically effective amount of one or more therapeutic agents other than the inhibitor or antagonist. The present invention also provides methods for treating cancer by administering a composition comprising an inhibitor or antagonist in combination with any other anti-cancer treatment (for example, checkpoint inhibitors, CAR-T cells, radiation therapy, chemotherapy or surgery) to a patient who has proven refractory to other treatments or who could benefit from adjunct therapy. The present invention also provides methods for the treatment of a patient having cancer and immunosuppressed by reason of having previously undergone one or more other cancer therapies. The present invention also provides alternative methods for the treatment of cancer where chemotherapy, radiation therapy, hormonal therapy, and/or biological therapy/immunotherapy has proven or can prove too toxic, for example, results in unacceptable or unbearable side effects, for the subject being treated.

### A. Subject

The subject can be any appropriate subject, including but not limited to human and non-human primates. Companion animals such but not limited to cats and dogs are also subjects. Test animals, anywhere from mice to humans are also considered subjects in the present invention.

### B. Types of Cancers to be Treated

Cancers that can be treated by the present invention include, but are not limited to, neoplasms, malignant tumors, metastases, or any disease or disorder characterized by uncontrolled cell growth such that it would be considered cancerous. The cancer can be a primary or metastatic cancer. Such cancers further include, but are not limited to, brain cancer including glioblastomas and medulloblastomas; biliary tract cancer; bladder cancer; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor. Commonly encountered cancers include breast, prostate, lung, colorectal, and brain cancer. In some embodiments the cancer is not stomach or gastrointestinal cancer.

Preferred cancers to be treated include but are not limited to glioblastoma, breast cancer, triple negative breast cancer, and a combination thereof.

Glioblastoma is the most common type of primary brain cancer. The cancer begins in astrocyte cells in the brain and is very fast-growing. The brain tumor cells can also suppress the immune system making it ineffective against the cancer. Therefore, people with glioblastoma or a variant called liposarcoma have poor long-term outcomes.

Breast cancer is a disease in which cells in the breast grow out of control and may form a tumor. There are different kinds of breast cancer. If the cancerous cells spread to other parts of the body, the disease is called metastatic breast cancer. About 12%, or 1 in 8, women in the U.S. will develop breast cancer during their lifetime. It is the second most common cancer among U.S. women, behind skin cancer. Breast cancer occurs mostly in women who are 50 years old or older, but men can also develop the disease. Each year in the U.S., about 245,000 cases of breast cancer are diagnosed in women and about 2,200 in men. Although many risk factors that increase a woman's chance of developing breast cancer have been identified, it is not yet known what causes normal cells to become cancerous. It is generally agreed that breast cancer is caused by a combination of genetic, hormonal, and environmental factors.

Triple-negative breast cancer is a kind of breast cancer that does not have any of the receptors that are commonly found in breast cancer. The first is for the female hormone, the second is for the female hormone, and the third is a protein called human epidermal growth factor (HER2).

The compounds and compositions of the present invention also can be administered to prevent progression to a neoplastic or malignant state. Such prophylactic use is indicated in conditions known or suspected of preceding progression to neoplasia or cancer, in particular, where non-neoplastic cell growth consisting of hyperplasia, metaplasia, or most particularly, dysplasia has occurred. Hyperplasia is a form of controlled cell proliferation involving an increase in cell number in a tissue or organ, without significant alteration in structure or function. Endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. A typical metaplasia involves a somewhat disorderly metaplastic epithelium. Dysplasia is frequently a forerunner of cancer and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

Alternatively, or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a patient, can indicate the desirability of prophylactic/therapeutic administration of the composition of the invention. Such characteristics of a transformed phenotype include morphology changes, looser substratum attachment, loss of contact inhibition, loss of anchorage dependence, protease release, increased sugar transport, decreased serum requirement, expression of fetal antigens.

In other aspects of the present invention, a patient which exhibits one or more of the following predisposing factors for malignancy is treated by administration of an effective amount of a composition of the invention: a chromosomal translocation associated with a malignancy (for example, the Philadelphia chromosome for chronic myelogenous leukemia t(9:22), t(14; 18) for follicular lymphoma, etc.), familial polyposis or Gardner's syndrome (possible forerunners of colon cancer), benign monoclonal gammopathy (a possible forerunner of multiple myeloma), a first degree kinship with persons having a cancer or precancerous disease showing a Mendelian (genetic) inheritance pattern (for example, familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; see Robbins and Angell, 1976, Basic Pathology, 2d Ed., W.B. Saunders Co., Philadelphia, pp. 112-113) etc.), and exposure to carcinogens (for example, smoking, and inhalation of or contacting with certain chemicals).

In one set of aspects of the present invention s, the invention includes a method of treating a subject susceptible to or exhibiting symptoms of cancer. The cancer can be primary, metastatic, recurrent or multi-drug resistant. In some cases, the cancer is drug-resistant or multi-drug resistant. As used herein, a “drug-resistant cancer” is a cancer that is resistant to conventional commonly known cancer therapies. Examples of conventional cancer therapies include treatment of the cancer with agents such as methotrexate, trimetrexate, adriamycin, taxotere, doxorubicin, 5-flurouracil, vincristine, vinblastine, pamidronate disodium, anastrozole, exemestane, cyclophosphamide, epirubicin, toremifene, letrozole, trastuzumab, megestrol, tamoxifen, paclitaxel, docetaxel, capecitabine, goserelin acetate, etc. A “multi-drug resistant cancer” is a cancer that resists more than one type or class of cancer agents, for example, the cancer is able to resist a first drug having a first mechanism of action, and a second drug having a second mechanism of action.

### C. Dose of Pharmaceutical Compositions

The active agents of the present invention can be administered to the subject in an effective amount for treating disorders such cancer.

An “pharmaceutically effective amount”, for instance, is an amount necessary or sufficient to realize a desired biologic effect. An “effective amount for autoimmune disease can be an amount sufficient to prevent or inhibit a decrease in T_{H} cells compared to the levels in the absence of peptide treatment. According to some aspects of the present invention, an effective amount is that amount of a compound of the invention alone or in combination with another medicament, which when combined or co-administered or administered alone, results in a therapeutic response to the disease, either in the prevention or the treatment of the disease. The biological effect can be the amelioration and or absolute elimination of symptoms resulting from the disease. In another embodiment, the biological effect is the complete abrogation of the disease, as evidenced for example, by the absence of a symptom of the disease.

The pharmaceutically effective amount of a compound of the present invention in the treatment of a disease described herein can vary depending upon the specific compound used, the mode of delivery of the compound, and whether it is used alone or in combination. The effective amount for any particular application can also vary depending on such factors as the disease being treated, the particular compound being administered, the size of the subject, or the severity of the disease or condition. One of ordinary skill in the art can empirically determine the effective amount of a particular molecule of the invention without necessitating undue experimentation. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject.

A pharmaceutically effective dosage should preferably produce a serum concentration of PI3Kinase antagonist and/or inhibitor of from about 0.1 ng/ml to about 50-100 μg/ml, for example. The pharmaceutical compositions typically should provide a dosage of from about 0.001 mg to about 2000 mg of PI3Kinase antagonist and/or inhibitor per kilogram of body weight per day, such as from about 0.01 mg to about 200 mg of PI3Kinase antagonist and/or inhibitor per kilogram of body weight per day, or from about 0.1 mg to about 20 mg of PI3Kinase antagonist and/or inhibitor per kilogram of body weight per day, or from about 1 mg to about 10 mg of PI3Kinase antagonist and/or inhibitor per kilogram of body weight per day, or from about 1 mg to about 5 mg of PI3Kinase antagonist and/or inhibitor per kilogram of body weight per day. Pharmaceutical dosage unit forms are prepared to provide from about 1 mg to about 1000 mg, such as from about 10 to about 500 mg of said active agent or a combination of said agents per dosage unit form.

A therapeutically effective dosage preferably produces a serum concentration of PI3Kinase antagonist and/or inhibitor of from about 0.1 ng/ml to about 50-100 μg/ml, for example. The pharmaceutical compositions typically should provide a dosage of from about 0.001 mg to about 2000 mg of PI3Kinase antagonist and/or inhibitor per kilogram of body weight per day, such as from about 0.01 mg to about 200 mg of PI3Kinase antagonist and/or inhibitor per kilogram of body weight per day, or from about 0.1 mg to about 20 mg of PI3Kinase antagonist and/or inhibitor per kilogram of body weight per day, or from about 1 mg to about 10 mg of PI3Kinase antagonist and/or inhibitor per kilogram of body weight per day, or from about 1 mg to about 5 mg of PI3Kinase antagonist and/or inhibitor per kilogram of body weight per day. Pharmaceutical dosage unit forms are prepared to provide from about 1 mg to about 1000 mg, such as from about 10 to about 500 mg of said active agent or a combination of said agents per dosage unit form.

Doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. The absolute amount will depend upon a variety of factors including the concurrent treatment, the number of doses and the individual patient parameters including age, physical condition, size and weight. These are factors well known to those of ordinary skill in the art and can be addressed with no more than routine experimentation. It is preferred generally that a maximum dose be used, that is, the highest safe dose according to sound medical judgment. Multiple doses of the molecules of the invention are also contemplated.

When used in combination with the therapies of the present invention the dosages of known therapies can be reduced in some instances, to avoid side effects.

In some aspects of the present invention, the present invention provides a method of treating a cancer comprising administering to a subject in whom such treatment is desired a therapeutically effective amount of a composition comprising an inhibitor or antagonist. A composition of the invention can, for example, be used as a first, second, third, or fourth line cancer treatment. In some aspects of the present invention, the invention provides methods for treating a cancer (including ameliorating a symptom thereof) in a subject refractory to one or more conventional therapies for such a cancer, said methods including administering to said subject a therapeutically effective amount of a composition comprising an inhibitor or antagonist. A cancer can be determined to be refractory to a therapy when at least some significant portion of the cancer cells are not killed or their cell division are not arrested in response to the therapy. Such a determination can be made either in vivo or in vitro by any method known in the art for assaying the effectiveness of treatment on cancer cells, using the art-accepted meanings of “refractory” in such a context. In a specific aspect of the present invention, a cancer is refractory where the number of cancer cells has not been significantly reduced or has increased.

### D. Route of Administration of Pharmaceutical Compositions

According to the methods of the present invention, the compounds and compositions can be administered in a pharmaceutical composition. In general, a pharmaceutical composition includes the compound of the invention and a pharmaceutically acceptable carrier.

“Locally administering” or “local administration” refers to administration (for example, by a subcutaneous, intramuscular, subdermal, intradermal, subcutaneous, intraperitoneally, intra-organ (for example injected into the cancerous tissue) or transdermal route), or any other route of administration, of a pharmaceutical agent to or to the vicinity of a target tissue, muscle or subdermal location by a non-systemic route. Thus, local administration excludes systemic (for example to the blood circulation system) routes of administration, such as intravenous or oral administration. Peripheral administration means administration to the periphery (for example to a location on or within a face, limb, trunk or head of a patient) as opposed to a visceral or gut (for example to the viscera) administration. In some embodiments local administration is administration to the site of the tumor, for instance, by intra-tumoral injection.

“Systemically administering” or “systemic administration” refers to administration (for example by an intravenous, inhalation, oral administration, or the like) of a pharmaceutical agent to or to the vicinity of a central tissue such as blood, lung or gut, that is remote from the local tissue that needs treatment. Thus, systemic administration excludes local administration to a diseased or damaged tissue. In some aspects of the present invention systemic administration is the administration to the blood circulation system.

The compounds of the present invention can be administered directly to a tissue. Direct tissue administration can be achieved by direct injection or other acceptable means and methods such as but not limited electroporation or direct contact with a tissue or organ. The compounds can be administered all at once, or alternatively they can be administered in a plurality of administrations. If administered multiple times, the compounds can be administered via different routes. For example, the first (or the first few) administrations can be made directly into the affected tissue while later administrations can be systemic.

Other agents when used in conjunction with the inhibitors of the invention can be delivered systemically.

Injectables are designed for local and systemic administration. Typically, a therapeutically effective dosage is formulated to contain a concentration of at least about 0.1% w/w up to about 90% w/w or more, such as more than 1% w/w of the PI3Kinase antagonist and/or inhibitor to the treated tissue(s). The PI3Kinase antagonist and/or inhibitor can be administered at once or can be divided into a number of smaller doses to be administered at intervals of time. It is understood that the precise dosage and duration of treatment is a function of the tissue being treated and can be determined empirically using known testing protocols or by extrapolation from in vivo or in vitro test data. It is to be noted that concentrations and dosage values can also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

Injectables are designed for local and systemic administration. Typically, a therapeutically effective dosage is formulated to contain a concentration of at least about 0.1% w/w up to about 90% w/w or more, such as more than 1% w/w of the PI3Kinase antagonist and/or inhibitor to the treated tissue(s). The PI3Kinase antagonist and/or inhibitor can be administered at once or can be divided into a number of smaller doses to be administered at intervals of time. It is understood that the precise dosage and duration of treatment is a function of the tissue being treated and can be determined empirically using known testing protocols or by extrapolation from in vivo or in vitro test data. It is to be noted that concentrations and dosage values can also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

### A. Regime of Pharmaceutical Compositions

The pharmaceutical composition of the present invention, such as but not limited to PI3Kinase antagonist and/or inhibitor can be administered at once or can be divided into a number of smaller doses to be administered at intervals of time. It is understood that the precise dosage and duration of treatment is a function of the disease state being treated and can be determined empirically using known testing protocols or by extrapolation from in vivo or in vitro test data. It is to be noted that concentrations and dosage values can also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the present invention.

The pharmaceutical composition of the present invention can be administered all at once or can be divided into a number of smaller doses to be administered at intervals of time. It is understood that the precise dosage and duration of treatment is a function of the disease state being treated and can be determined empirically using known testing protocols or by extrapolation from in vivo or in vitro test data. It is to be noted that concentrations and dosage values can also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed methods.

The compounds and compositions of the invention can be administered directly to a tissue. Direct tissue administration can be achieved by direct injection. The compounds and compositions can be administered once, or alternatively they can be administered in a plurality of administrations. If administered multiple times, the compounds and compositions can be administered via different routes. For example, the first (or the first few) administrations can be made directly into the affected tissue while later administrations can be systemic.

### B. Formulations of Pharmaceutical Compositions

According to the methods of the present invention, the pharmaceutical composition of the present invention can be administered to a subject in a pharmaceutical composition. In general, a pharmaceutical composition includes the compound of the invention and a pharmaceutically acceptable carrier.

The formulations of the present invention are administered in pharmaceutically acceptable solutions, which can routinely contain pharmaceutically acceptable concentrations of salt, buffering agents, preservatives, compatible carriers, adjuvants, and optionally other therapeutic ingredients.

Pharmaceutical compositions of the present invention include an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (including human and non-human subjects) administration, it will be understood that preparations should meet sterility, pyrogenicity, general safety and purity standards as required by FDA Office of Biological Standards. The compounds and compositions of the present invention are generally suitable for administration to humans and non-humans. Preferably, a compound or composition is nontoxic and sufficiently pure so that no further manipulation of the compound or composition is needed prior to administration to humans.

Pharmaceutical compositions of the present invention include an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (for example, human and non-human) administration, it will be understood that preparations should meet sterility, pyrogenicity, general safety and purity standards as required by FDA Office of Biological Standards. The compounds are generally suitable for administration to humans and non-humans. This term requires that a compound or composition be nontoxic and sufficiently pure so that no further manipulation of the compound or composition is needed prior to administration to humans.

As used herein, “pharmaceutically acceptable carrier” includes any and all solvents, dispersion media, coatings, surfactants, antioxidants, preservatives (for example, antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated.

As used herein, “pharmaceutically acceptable carrier” includes any and all solvents, dispersion media, coatings, surfactants, antioxidants, preservatives (for example, antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated.

Pharmaceutically acceptable carriers include but are not limited to diluents, fillers, salts, buffers, stabilizers, solubilizers and other materials which are well-known in the art. Exemplary pharmaceutically acceptable carriers for peptides in particular are described in U.S. Pat. No. 5,211,657. Such preparations can routinely contain salt, buffering agents, preservatives, compatible carriers, and optionally other therapeutic agents. When used in medicine, the salts should be pharmaceutically acceptable, but non-pharmaceutically acceptable salts can conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts.

Typically, by way of example and without limitation, the compositions of the present invention are preferably formulated for a single dosage administration such as from a single container or pill rather than multiple doses, though multiple such doses are included within the scope of the invention. To formulate a composition of the present invention, the weight fraction of PI3Kinase inhibitor and/or agonist or other composition of the present invention is dissolved, suspended, dispersed or otherwise mixed in a selected vehicle at an effective concentration such that the treated condition is relieved or ameliorated. Pharmaceutical carriers or vehicles suitable for administration of a composition such as PI3Kinase inhibitor and/or agonist can include any such carriers known to those skilled in the art to be suitable for the particular mode of administration.

The agent can include different types of carriers depending on whether it is to be administered in solid, liquid or aerosol form, and whether it need to be sterile for such routes of administration as injection. The present invention can be administered intravenously, intradermally, intraarterially, intralesionally, intratumorally, intracranially, intraarticularly, intraprostatically, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (for example, aerosol inhalation), injection, infusion, continuous infusion, localized perfusion bathing target cells directly, via a catheter, via a lavage, in cremes, in lipid compositions (for example, liposomes), or by other method or any combination of the forgoing as would be known to one of ordinary skill in the art (see, for example, Remington's Pharmaceutical Sciences (1990), incorporated herein by reference). In a particular embodiment of the present invention, intraperitoneal injection is contemplated.

The pharmaceutical composition of the present invention can include various antioxidants to retard oxidation of one or more components. Additionally, the prevention of the action of microorganisms can be brought about by preservatives such as various antibacterial and antifungal agents, including but not limited to parabens (for example, methylparabens, propylparabens), chlorobutanol, phenol, sorbic acid, thimerosal or combinations thereof.

The agent can be formulated into a composition in a free base, neutral or salt form. Pharmaceutically acceptable salts, include the acid addition salts, for example, those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In certain aspects of the present invention where the composition is in a liquid form, a carrier can be a solvent or dispersion medium comprising but not limited to, water, ethanol, polyol (for example, glycerol, propylene glycol, liquid polyethylene glycol, polyethylene glycol, etc.), lipids (for example, triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

A compound or composition of the present invention can be used directly or can be mixed with suitable adjuvants and/or carriers. Suitable adjuvants include aluminum salt adjuvants, such as aluminum phosphate or aluminum hydroxide, calcium phosphate nanoparticles (BioSante Pharmaceuticals, Inc.), ZADAXIN™, iron oxide nanoparticles, lipid nanoparticles, nucleotides ppGpp and pppGpp, killed Bordetella pertussis or its components, Corenybacterium derived P40 component, cholera toxin and mycobacteria whole or parts, and ISCOMs (DeVries et al., 1988; Morein et al., 199&, Lovgren: al., 1991). Also useful as adjuvants are Pam3Cys, LPS, ds and ss RNA. The skilled artisan is familiar with carriers appropriate for pharmaceutical use or suitable for use in humans.

The composition of the invention can be administered in various ways and to different classes of recipients.

The compounds and compositions of the present invention can be formulated into preparations in solid, semi-solid, liquid or gaseous forms such as tablets, capsules, powders, granules, ointments, solutions, depositories, inhalants and injections, and usual ways for oral, parenteral or surgical administration. The present invention also embraces pharmaceutical compositions which are formulated for local administration, such as by implants.

Compounds and compositions of the present invention can be dissolved, suspended, dispersed or otherwise mixed in a selected vehicle at an effective concentration such that the treated condition is relieved or ameliorated. Pharmaceutical carriers or vehicles suitable for administration of the PI3Kinase antagonist and/or inhibitor include any such carriers known to those skilled in the art to be suitable for the particular mode of administration.

In any case, the composition and compounds can include various antioxidants to retard oxidation of one or more components. Additionally, the prevention of the action of microorganisms can be brought about by preservatives such as various antibacterial and antifungal agents, including but not limited to parabens (for example, methylparabens, propylparabens), chlorobutanol, phenol, sorbic acid, thimerosal or combinations thereof.

The agent can be formulated into a composition in a free base, neutral or salt form. Pharmaceutically acceptable salts, include the acid addition salts, for example, those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In embodiments of the present invention where the composition is in a liquid form, a carrier can be a solvent or dispersion medium comprising but not limited to, water, ethanol, polyol (for example, glycerol, propylene glycol, liquid polyethylene glycol, etc.), lipids (for example, triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; polyethylene glycol (PEG), or combinations thereof such methods. In many cases, it will be preferable to include isotonic agents, such as, for example, sugars, sodium chloride or combinations thereof.

The compounds and compositions of the invention can be used directly or can be mixed with suitable adjuvants and/or carriers. Suitable adjuvants include aluminum salt adjuvants, such as aluminum phosphate or aluminum hydroxide, calcium phosphate nanoparticles (BioSante Pharmaceuticals, Inc.), ZADAXIN™, nucleotides ppGpp and pppGpp, killed Bordetella pertussis or its components, Corynebacteriu, derived P40 component, cholera toxin and mycobacteria whole or parts, and ISCOMs (DeVries et al., 1988; Morein et al., 199&, Lovgren: al., 1991). Also useful as adjuvants are polyinosinic: polycytidylic acid (poly I:C) Pam3Cys, LPS, ds and ss RNA. The skilled artisan is familiar with carriers appropriate for pharmaceutical use or suitable for use in humans.

The composition of the invention can be administered in various ways and to different classes of recipients.

The formulations of the present invention are administered in pharmaceutically acceptable solutions, which can routinely contain pharmaceutically acceptable concentrations of salt, buffering agents, preservatives, compatible carriers, adjuvants, and optionally other therapeutic ingredients.

Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. Such preparations can routinely contain salt, buffering agents, preservatives, compatible carriers, and optionally other therapeutic agents. When used in medicine, the salts should be pharmaceutically acceptable, but non-pharmaceutically acceptable salts can conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts.

The compounds and compositions of the present invention can be formulated into preparations in solid, semi-solid, liquid, or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Compounds and compositions suitable for oral administration can be presented as discrete units, such as capsules, tablets, lozenges, each containing a predetermined amount of the active agent. Other compositions include suspensions in aqueous liquids or non-aqueous liquids, such as a syrup, an elixir or an emulsion.

Other agents when used in conjunction with the inhibitors of the invention can be delivered systemically.

For oral administration, the compositions and compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate. Optionally the oral formulations can also be formulated in saline or buffers for neutralizing internal acid conditions or can be administered without any carriers.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Microspheres formulated for oral administration can also be used. Such microspheres have been well defined in the art. All formulations for oral administration should be in dosages suitable for such administration.

For buccal administration, the compositions can take the form of tablets or lozenges formulated in conventional manner.

For administration by inhalation, the compounds for use according to the present invention can be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of for example gelatin for use in an inhaler or insufflator can be formulated containing a powder mix of the compound and a suitable powder base such as lactose or starch. Techniques for preparing aerosol delivery systems are well known to those of skill in the art. Generally, such systems should utilize components which will not significantly impair the biological properties of the active agent (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds and compositions of the present invention, when it is desirable to deliver them systemically, can be formulated for parenteral administration by injection, for example, by bolus injection or continuous infusion. Formulations for injection can be presented in unit dosage form, for example, in ampoules or in multi-dose containers, with an added preservative. The compounds and compositions can take such forms as suspensions, solutions or emulsions in oily or aqueous vehicles, and can contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, antioxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) can be employed to the extent that patient tolerance permits. Multiple doses per day are contemplated to achieve appropriate systemic levels of compounds.

The pharmaceutical compositions of the present invention can be administered in combination with other therapeutic agents and such administration can be simultaneous or sequential, either before or after the other therapeutic. When the other therapeutic agents are administered simultaneously, they can be administered in the same or separate formulations, but are administered at the same time, or at different time, or a combination thereof. The administration of the other therapeutic agent and the inhibitor or antagonist can also be temporally separated, meaning that the therapeutic agents are administered at a different time, either before or after, the administration of the inhibitor or antagonist. The separation in time between the administration of these compounds can be a matter of minutes or it can be longer.

Preparations for parenteral administration include but are not limited to sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, antioxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) can be employed to the extent that patient tolerance permits. Multiple doses per day are contemplated to achieve appropriate systemic levels of compounds.

Compositions suitable for oral administration can be presented as discrete units, such as capsules, tablets, lozenges, each containing a predetermined amount of the active agent. Other compositions include suspensions in aqueous liquids or non-aqueous liquids, such as a syrup, an elixir or an emulsion.

For oral administration, the compounds and compositions of the present invention can be formulated readily by combining the active compounds with pharmaceutically acceptable carriers well known in the art. Such carriers allow the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, include but are not limited to, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate. Optionally the oral formulations can also be formulated in saline or buffers for neutralizing internal acid conditions or can be administered without any carriers.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Microspheres formulated for oral administration can also be used. Such microspheres have been well defined in the art. All formulations for oral administration should be in dosages suitable for such administration.

For buccal administration, the compositions can take the form of tablets or lozenges formulated in conventional manner.

For administration by inhalation, the compounds and compositions of the present invention for use according to the present invention can be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of for example gelatin for use in an inhaler or insufflator can be formulated containing a powder mix of the compound and a suitable powder base such as lactose or starch.

Techniques for preparing aerosol delivery systems are well known to those of skill in the art. Generally, such systems can utilize components that will not significantly impair the biological properties of the active agent (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds and compositions of the present invention, when it is desirable to deliver them systemically, can be formulated for parenteral administration by injection, for example, by bolus injection or continuous infusion. Formulations for injection can be presented in unit dosage form, for example, in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

### C. Articles of Manufacture, Packaging, Storage, and Kits for Pharmaceutical Compositions

The present invention also includes articles of manufacture, which refers to any one or collection of components. In some aspects of the present invention the articles include kits. The articles include pharmaceutical or diagnostic grade compounds of the invention in one or more containers. The article can include instructions or labels promoting or describing the use of the compounds of the invention.

As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention in connection with treatment of disease.

“Instructions” can include a component of promotion, and typically involve written instructions on or associated with packaging of compositions of the invention. Instructions also can include any oral or electronic instructions provided in any manner.

Thus, the agents described herein can, in some embodiments of the present invention, be assembled into pharmaceutical or diagnostic or research kits to facilitate their use in therapeutic, diagnostic or research applications. A kit can include one or more containers housing the components of the invention and instructions for use. Specifically, such kits can include one or more agents described herein, along with instructions describing the intended therapeutic application and the proper administration of these agents. In certain embodiments of the present invention agents in a kit can be in a pharmaceutical formulation and dosage suitable for a particular application and for a method of administration of the agents.

The kit can be designed to facilitate use of the methods described herein by physicians and can take many forms. Each of the compositions of the kit, where applicable, can be provided in liquid form (for example, in solution), or in solid form, (for example, a dry powder). In certain cases, some of the compositions can be constitutable or otherwise processable (for example, to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (for example, videotape, DVD, etc.), Internet, and/or web-based communications, etc. The written instructions can be in a form prescribed by a governmental agency regulating the manufacture, use or sale of pharmaceuticals or biological products, which instructions can also reflect approval by the agency of manufacture, use or sale for human administration.

The kit can contain any one or more of the components described herein in one or more containers. As an example, in one embodiment, the kit can include instructions for mixing one or more components of the kit and/or isolating and mixing a sample and applying to a subject. The kit can include a container housing agents described herein. The agents can be prepared sterilely, packaged in syringe and shipped refrigerated. Alternatively, a kit can be housed in a vial or other container for storage. A second container can have other agents prepared sterilely. Alternatively, the kit can include the active agents premixed and shipped in a syringe, vial, tube, or other container.

The kit can have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box or a bag. The kit can be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit can also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The compositions of the kit can be provided as any suitable form, for example, as liquid solutions or as dried powders. When the composition provided is a dry powder, the powder can be reconstituted by the addition of a suitable solvent, which can also be provided. In embodiments where liquid forms of the composition are sued, the liquid form can be concentrated or ready to use. The solvent can depend on the compound and the mode of use or administration. Suitable solvents for drug compositions are well known and are available in the literature. The solvent can depend on the compound and the mode of use or administration. The kits, in one set of embodiments, can include a carrier means being compartmentalized to receive in close confinement one or more container means such as vials, tubes, and the like, each of the container means comprising one of the separate elements to be used in the method. For example, one of the containers can include a positive control for an assay. Additionally, the kit can include containers for other components, for example, buffers useful in the assay.

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient is sterile and suitable for administration as a particulate free solution. In other words, the present invention encompasses both parenteral solutions and lyophilized powders, each being sterile, and the latter being suitable for reconstitution prior to injection. Alternatively, the unit dosage form can be a solid suitable for oral, transdermal, topical or mucosal delivery.

In a preferred embodiment of the present invention, the unit dosage form is suitable for intravenous, intramuscular or subcutaneous delivery. Thus, the present invention encompasses solutions, preferably sterile, suitable for each delivery route.

In another preferred embodiment of the present invention, compositions and compositions of the present invention are stored in containers with biocompatible detergents, including but not limited to, lecithin, taurocholic acid, and cholesterol; or with other proteins, including but not limited to, gamma globulins and serum albumins. More preferably, compositions of the invention can be stored with human serum albumins for human uses, and stored with bovine serum albumins for veterinary uses.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures (such as methods for monitoring mean absolute lymphocyte counts, tumor cell counts, and tumor size) and other monitoring information.

More specifically, the present invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material. The present invention also provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. The invention further provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. The present invention further provides an article of manufacture comprising a needle or syringe, preferably packaged in sterile form, for injection of the formulation, and/or a packaged alcohol pad.

The present invention also includes articles of manufacture, which refers to any one or collection of components. In some embodiments the articles are kits. The articles include pharmaceutical or diagnostic grade compounds of the invention in one or more containers. The article can include instructions or labels promoting or describing the use of the compounds of the present invention.

As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the present invention in connection with treatment of disease.

“Instructions” can define a component of promotion, and typically involve written instructions on or associated with packaging of compositions of the invention. Instructions also can include any oral or electronic instructions provided in any manner.

Thus, the agents described herein can, in some aspects of the present invention, be assembled into pharmaceutical or diagnostic or research kits to facilitate their use in therapeutic, diagnostic or research applications. A kit can include one or more containers housing the components of the invention and instructions for use. Specifically, such kits can include one or more agents described herein, along with instructions describing the intended therapeutic application and the proper administration of these agents. In certain aspects of the present invention agents in a kit can be in a pharmaceutical formulation and dosage suitable for a particular application and for a method of administration of the agents.

The kit can be designed to facilitate use of the methods described herein by physicians and can take many forms. Each of the compositions of the kit, where applicable, can be provided in liquid form (for example, in solution), or in solid form, (for example, a dry powder). In certain cases, some of the compositions can be constitutable or otherwise processable (for example, to an active form), for example, by the addition of a suitable solvent or other species (for example, water or a cell culture medium), which may or may not be provided with the kit.

As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (for example, videotape, DVD, etc.), Internet, and/or web-based communications, etc. The written instructions can be in a form prescribed by a governmental agency regulating the manufacture, use or sale of pharmaceuticals or biological products, which instructions can also reflect approval by the agency of manufacture, use or sale for human administration.

The kit can include any one or more of the components described herein in one or more containers. As an example, the kit can include instructions for mixing one or more components of the kit and/or isolating and mixing a sample and applying to a subject. The kit can include a container housing agents described herein. The agents can be prepared sterilely, packaged in syringe and shipped refrigerated. Alternatively, it can be housed in a vial or other container for storage. A second container can have other agents prepared sterilely. Alternatively, the kit can include the active agents premixed and shipped in a syringe, vial, tube, or other container.

The kit can have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box or a bag. The kit can be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit can also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The compositions of the kit can be provided as any suitable form, for example, as liquid solutions or as dried powders. When the composition provided is a dry powder, the powder can be reconstituted by the addition of a suitable solvent, which can also be provided. In embodiments where liquid forms of the composition are sued, the liquid form can be concentrated or ready to use. The solvent will depend on the compound and the mode of use or administration. Suitable solvents for drug compositions are well known and are available in the literature. The solvent will depend on the compound and the mode of use or administration.

The kits, in one set of aspects of the present invention, can include a carrier means being compartmentalized to receive in close confinement one or more container means such as vials, tubes, and the like, each of the container means comprising one of the separate elements to be used in the method. For example, one of the containers can include a positive control for an assay. Additionally, the kit can include containers for other components, for example, buffers useful in the assay.

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient is sterile and suitable for administration as a particulate free solution. In other words, the invention encompasses both parenteral solutions and lyophilized powders, each being sterile, and the latter being suitable for reconstitution prior to injection. Alternatively, the unit dosage form can be a solid suitable for oral, transdermal, topical or mucosal delivery.

In a preferred aspect of the present invention, the unit dosage form is suitable for intravenous, intramuscular or subcutaneous delivery. Thus, the invention encompasses solutions, preferably sterile, suitable for each delivery route.

In another preferred embodiment of the present invention, compositions of the invention are stored in containers with biocompatible detergents, including but not limited to, lecithin, taurocholic acid, and cholesterol; or with other proteins, including but not limited to, gamma globulins and serum albumins. More preferably, compositions of the invention are stored with human serum albumins for human uses and stored with bovine serum albumins for veterinary uses.

As with any pharmaceutical product, the packaging material and container are designed to protect the stability of the product during storage and shipment. Further, the products of the present invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures (such as methods for monitoring mean absolute lymphocyte counts, tumor cell counts, and tumor size) and other monitoring information.

More specifically, the present invention provides an article of manufacture including packaging material, such as a box, bottle, tube, vial, container, sprayer, insufflator, intravenous (i.v.) bag, envelope and the like; and at least one unit dosage form of a pharmaceutical agent contained within said packaging material. The invention also provides an article of manufacture including packaging material, such as a box, bottle, tube, vial, container, sprayer, insufflator, intravenous (i.v.) bag, envelope and the like; and at least one unit dosage form of each pharmaceutical agent contained within said packaging material. The invention further provides an article of manufacture including packaging material, such as a box, bottle, tube, vial, container, sprayer, insufflator, intravenous (i.v.) bag, envelope and the like; and at least one unit dosage form of each pharmaceutical agent contained within said packaging material. The invention further provides an article of manufacture including a needle or syringe, preferably packaged in sterile form, for injection of the formulation, and/or a packaged alcohol pad.

VII PI3K Inhibitors with Checkpoint Inhibitors for Treating Cancer-Methods of Treating Glioblastoma, Breast Cancer, and Triple Negative Breast Cancer

The present invention also includes methods of treating glioblastoma, breast cancer, and/or triple negative breast cancer.

A seventh aspect of the present invention includes a method of treating a subject with cancer, including but not limited to: a) providing a subject in need of treatment for at least one cancer; wherein the cancer is glioblastoma, breast cancer, triple negative breast cancer, or a combination thereof; b) providing at least one pharmaceutical composition including: 1) at least one PI3K inhibitor in a pharmaceutically effective amount in a pharmaceutically acceptable carrier; and 2) at least one checkpoint inhibitor in a pharmaceutically effective amount in a pharmaceutically acceptable carrier; c) administering a pharmaceutically effective amount of the at least one pharmaceutical composition to said subject; wherein the subject is treated for the at least one cancer.

In an aspect of the present invention, the method further includes but is not limited to administering at least one treatment to the subject.

In another aspect of the present invention, the treatment includes but is not limited to at least one chemotherapy, at least one immunotherapy, at least one radiation, at least one low temperature, at least one high temperature, at least one laser, at least one surgery, at least one nanoparticle delivery system, or a combination thereof.

In a further aspect of the present invention, the at least one PI3K inhibitor and the checkpoint inhibitor are provided together or separately.

In an aspect of the present invention, the at least one PI3K inhibitor includes but is not limited to:

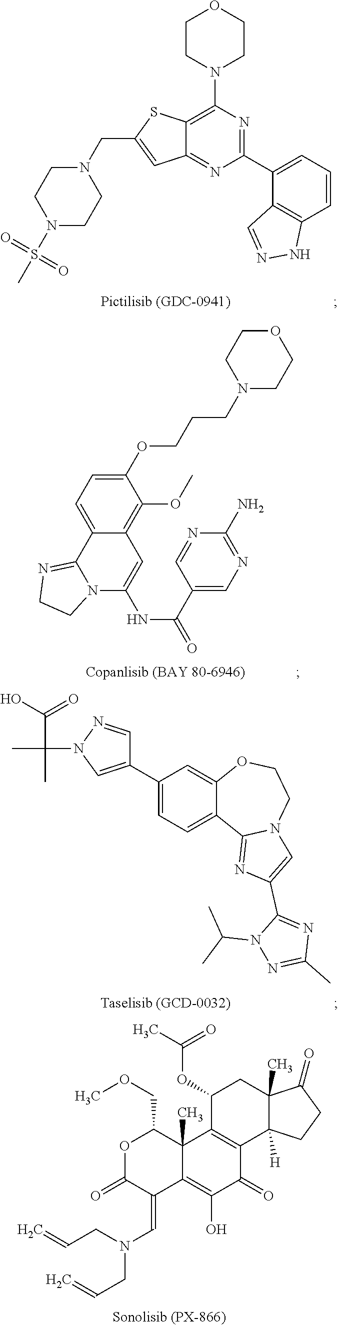

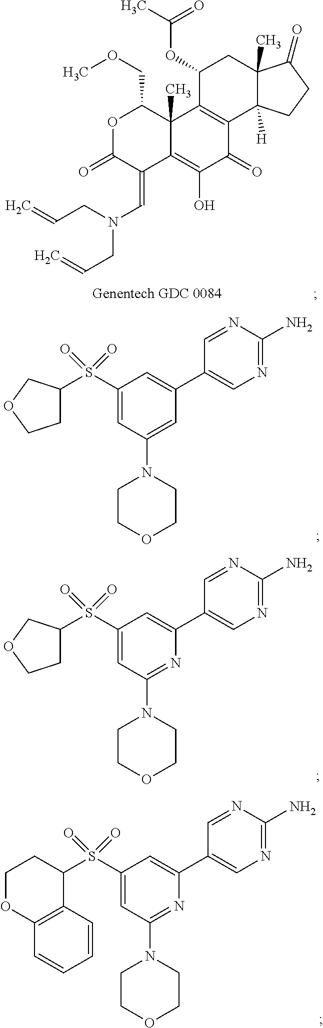 [SMILES: C=CCN(/C=C1\C(=O)O[C@H](COC)[C@@]2(C)C1=C(O)C(=O)C1=C2[C@H](OC(C)=O)C[C@]2(C)C(=O)CC[C@@H]12)CC=C ; Nc1ncc(-c2cc(N3CCOCC3)cc(S(=O)(=O)C3CCOC3)c2)cn1 ; Nc1ncc(-c2cc(S(=O)(=O)C3CCOC3)cc(N3CCOCC3)n2)cn1 ; Nc1ncc(-c2cc(S(=O)(=O)C3CCOc4ccccc43)cc(N3CCOCC3)n2)cn1]

or a combination thereof.

In another aspect of the present invention, the at least one checkpoint inhibitor includes but is not limited to: a) at least one anti-CTLA-4 inhibitor; b) at least one PD-1 inhibitor; c) at least one anti-PD-L1 inhibitor; or d) a combination thereof.

In a further aspect of the present invention the anti-CTLA-4 inhibitor includes but is not limited to: ipilimumab.

In an additional aspect of the present invention, the PD-1 inhibitor includes but is not limited to pembrolizumab, nivolumab, cemiplimab (trade name LIBTAYO®), and dostarlimab (trade name JEMPERLI®), or a combination thereof.

In an aspect of the present invention, the anti-PD-L1 inhibitor includes but is not limited to atezolizumab (trade name TECENTRIQ®), durvalumab (trade name IMFINZI®), and Avelumab (trade name BAVENCIO®), or a combination thereof.

### EXAMPLES

### Example 1: Glioblastoma

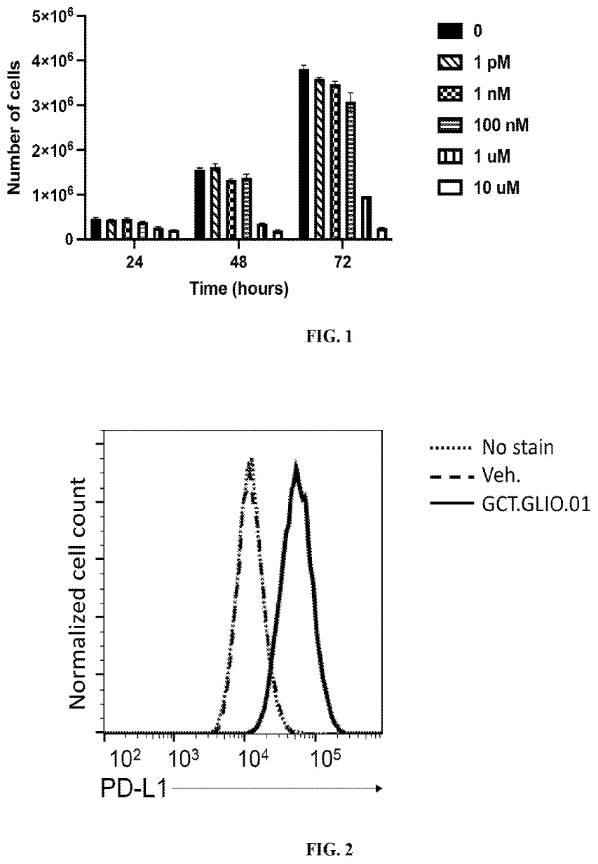

This example refers to FIG. 1 through FIG. 3.

The purpose of these experiments was to address how, and if, inhibitors of PI3 kinase, affect glioblastoma (GBM) cells grown in culture. GL261 mouse glioblastoma cells were cultured in tissue culture plates at an approximate concentration of 5×10⁵ cells/ml of complete RPMI culture medium containing 5% heat inactivated fetal calf serum, L-glutamine, HEPES buffer, and an antibiotic cocktail of penicillin and streptomycin. Cells were treated with several doses of GCT.Glio.1 ranging from 10 nanoMolar to 10 microMolar final concentrations for 48 hours. Cells were then harvested and counted. The first experiments showed that treatment with the compound resulted in many fewer cells, as indicated by cell counting, after treatment than the number of cells in the untreated group. The greatest reduction in cell number occurred at 10 microMolar final concentration of the Glio.1 (also known as GCT0007) compound, as illustrated in FIG. 1. Quantification of cell viability shows that remaining cells were live with no evidence of cell death, suggesting that the compound reduced the number of responding cells, most likely as a result of growth arrest of the rapidly dividing GL261 cells. Ongoing studies have confirmed that the compounds cause cell cycle arrest at the G2/M cell cycle checkpoint.

The second step in the study was to determine if the compound changed the way the GBM cells in culture “look” to the immune system. In other words, does treatment with GCT increase the likelihood of T cells getting “revved up” and releasing the “brakes” on an anti-tumor response. In this experiment, cells were cultured at 5×10⁵ cells/ml of complete RPMI culture medium containing 5% heat inactivated fetal calf serum, L-glutamine, HEPES buffer, and an antibiotic cocktail of penicillin and streptomycin. Cells were treated with 1 microgram/ml GCT.Glio.1 (also known as GCT07) for 48 hours. Cells were then harvested, counted, and stained using fluorochrome-conjugated antibodies to mouse MHC class II and mouse PD-L1. Cells were then harvested washed and analyzed using a flow cytometry. The data show that the GCT treatment increases PD-L1 expression, the target of PD-1: PD-L1 targeted immune check point inhibitors, on the cell surface of the GBM, potentially providing a target for “releasing the brakes,” by treating the patient with a “checkpoint inhibitor”. In the following experiments, we demonstrate that treatment with either compound increases MHC Class II, FIG. 2.

FIG. 1. GL261 mouse GBM cells were cultured for 24, 48 or 72 hours in the presence GCT.GLIO.1 at the indicated concentrations. Cells were then harvested and counted.

FIG. 2. GL261 cells were cultured with and without GCT.GLIO.1 (also known as GCT007), for 72 hours, harvested, and stained with anti-PD-L1 as shown. The histograms indicate an increase in the cell surface expression of PD-L1 corresponding to an increased dose of GCT.GLIO.1 and indicate that increased PD-L1 can provide a target for immune checkpoint inhibitor therapy.

FIG. 3. GL261 cells were cultured with and without GCT.GLIO.1, as indicated, for 7 days, harvested, and stained as indicated to determine changes in the cell surface expression of PD-L1 and MHC class II; or for the expression of a marker of autophagy. LC3. The increases in MHC class II and PD-L1 suggest that the drug would increase the likelihood of recognition by CD4 T cells and that increased PD-L1 would provide a target for immune checkpoint inhibitor therapy

Results: These results show that both compounds increase the level of MHC class II, but compound GCT.GLIO.1 (NPT520-337) caused increases in all three molecules, whereas compound GCT.GLIO.2 (NPT520-322) causes growth arrest and increased MHC class II expression.

### Example 2: Glioblastoma

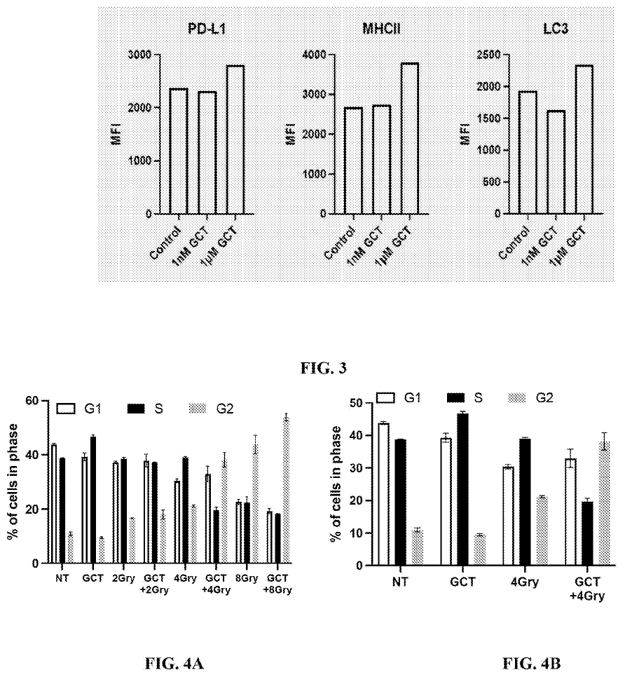

This example refers to FIG. 4A and FIG. 4B through FIG. 6.

Effects of GCT.Glio.1 on Radiation-sensitivity of GL261. In these experiments, we addressed the question of how, or if, treatment with GCT.Glio.1 can affect subsequent treatment with irradiation, FIG. 4A and FIG. 4B. GL261 cells were cultured with and without GCT.GLIO.1 (NPT520-337) denoted GCT at a concentration of 1 microgram/ml for 48 hours, with or without treatment with 2 Gy irradiation (denoted as R). Cells were then harvested, and viability of the recovered cells was assessed using Trypan Blue viability dye. The viability data indicate that GCT.Glio.1 sensitizes the cells to 2 Gy as indicated by lower viability (increased cell death) following GCT and irradiation, FIG. 4A and FIG. 4B.

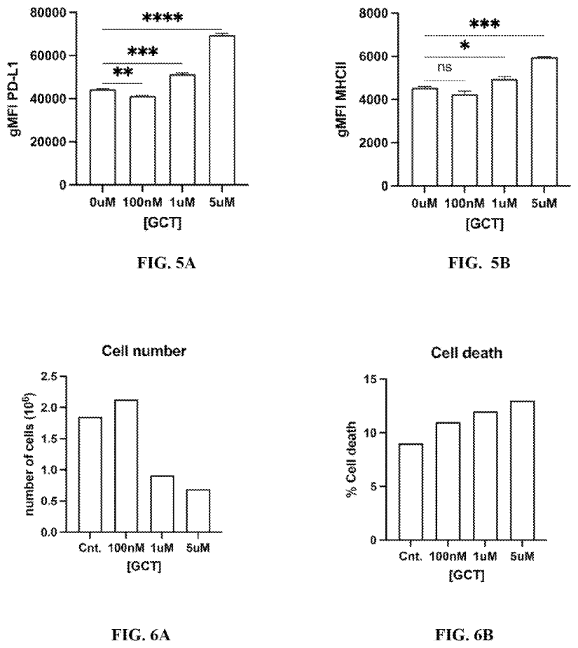

In addition to inducing growth arrest, GCT.GLIO.1 increased cell surface PD-L1 and MHC class II (denoted as Mean Fluorescence Intensity (MFI)), potentially making these cells a better target for immunotherapy (FIG. 5).

Similar results were found when human U251 glioma cells were treated with increasing doses of GCT.GLIO.1. We found that GCT.GLIO.1 induced growth arrest and cell death in these cells in a dose-dependent manner (FIG. 6)

There are several isoform specific PI3K inhibitors in early-stage clinical trials. A key problem has been drug delivery beyond the blood brain barrier, however Genentech's GDC 0084 is an orally available small molecule PI3K inhibitor that can permeate the blood brain barrier.

The compounds designated GCT.GLIO.X were also designed to be brain permeant. The novel aspects of GCT.GLIO.1 and GCT.GLIO.2 are the combined advantages of being able to cross the blood brain barrier and to alter the “visibility” of the GBM to the immune system.

FIG. 4A and FIG. 4B. Effects of GCT.GLIO.1 on radiation sensitivity and Cell Cycle Arrest of GL261. In the next experiment, we sought to identify the optimum dose of radiation following treatment with GCT.Glio.1 to achieve cell cycle arrest. In these studies, we treated GL261 cells cultured at 5×10⁵ cells/ml with 1 microgram/ml GCT.Glio.1 for 24 hours followed by increasing doses of irradiation, including 2, 4, and 8 Gy. Cells were then harvested and counted. The cell cycle stage of the recovered cells was determined by mild detergent solubilization with saponin, followed by staining with propidium iodide, and followed by light fixation in paraformaldehyde. The stained cells were analyzed by flow cytometry programmed to determine the frequency of cells arrested at each stage of the cell cycle. The results show that retreatment with GCT.Glio.1 significantly amplified the response to radiation and caused significant increase in cells arrested at the G2 cell cycle checkpoint.

FIG. 5A and FIG. 5B. Human GBM cell line U251 were cultured at 5×10⁵ cells/ml, of complete RPMI culture medium containing 5% heat inactivated fetal calf serum, L-glutamine, HEPES buffer, and an antibiotic cocktail of penicillin and streptomycin, treated with 1 microgram/ml GCT.Glio.1, harvested, counted, and stained with fluorochrome-conjugated antibodies to PD-L1 or MHC class II to determine changes in cell surface PD-L1, left panel, and MHC class II, right panel.

FIGS. 6A and 6B. Human U251 cell lines were cultured at 5×10⁵ cells/ml, of complete RPMI culture medium containing 5% heat inactivated fetal calf serum, L-glutamine, HEPES buffer, and an antibiotic cocktail of penicillin and streptomycin. The cells were treated with 1 microgram/ml GCT.Glio.1, harvested, counted, and assessed for changes in cell number and in dead cells.

### Example 3: Breast Cancer

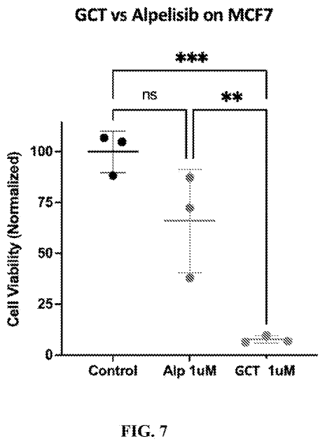

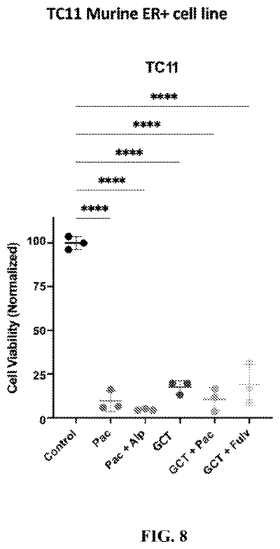

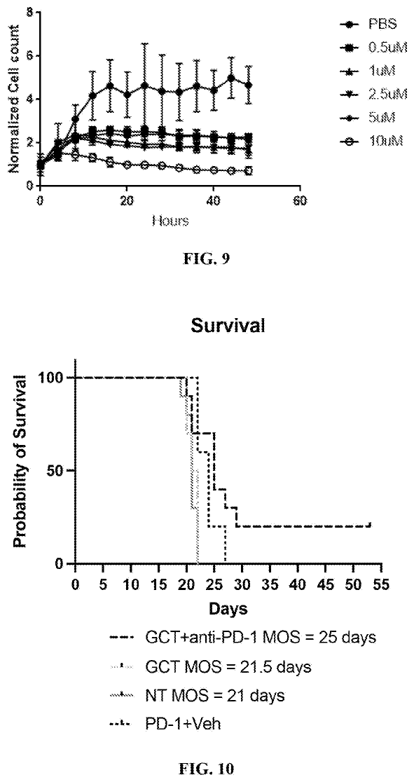

This example refers to FIG. 7, FIG. 8, and FIG. 9.

Breast cancer is a well-documented serious public health threat that despite huge efforts remains an active area of research and development for efficacious treatments, notably as there are drug resistant variants of breast cancer that remain notably problematic. Despite decades of therapeutic trials, improved diagnostics, new therapies, and hundreds, if not thousands, of breast cancer clinical trials, breast cancer remains the deadliest form of cancer in women. The innovation and excitement for immunotherapy for many tumors has not been successful for breast cancers because the tumor is immunologically silent or “cold”.

In the present application, GCT.BC.1 (also known as GCT007, GCT.GLIO.1, NPT520-337) has been found to be a novel, small molecule PI3Kinas inhibitor, and that GCT.BC.1 can turn an immunologically “cold” tumor to one that expresses many of the target molecules for immune therapies, thus turning the tumor from “cold” to “hot” with regards to immune therapy. Radiation can penetrate deeply even through bone, it can cover small or large areas, and low doses are comparatively non-toxic. Thus, a radiation activatable prodrug would also reduce the amount of radiation needed to control tumors and thereby significantly diminish normal tissue damage. If the prodrug were sequestered, and washed out of normal tissues, the drug could be delivered with much less danger to normal peripheral tissues.

These examples relate to in vitro human and mouse cell line data in breast cancer. These experiments were designed to test the impact of GCT.BC.1 on breast cancer. These data show GCT.BC.1 is as effective at killing and preventing cellular proliferation as other PI3K inhibitors and can be effectively combined with other approved chemotherapeutic agents.

FIG. 7. Cell viability of human MCF7 breast cancer cells. Human MCF7 cells were treated with 1 uM Apelisib (Alp), 1 uM GCT.BC.1 (also known as GCT007, GCT.GLIO.1, NPT520-337), or DMSO control for 48 hours. Viability was determined by cleaved caspase-7 and was measured using a Caspase Glo assay (Promega) as described by the manufacturer.

FIG. 8, Cell viability of mouse TC11 breast cancer cells. TC11 cells were treated with 100 nM Paclitaxel (Pac), 100 nM Paclitaxel+1 uM Alpelisib (Alp), 0.50 uM GCT.BC.1 (GCT), 0.50 uM GCT.Glio.1+100 nM Paclitaxel, 0.50 uM GCT.BC.1+7.5 nM Fulvestrant, or DMSO control as indicated for 48 hours. Viability was determined by cleaved caspase-7 and was measured using a Caspase Glo assay (Promega) as described by the manufacturer.

FIG. 9, Proliferation of 66CL4 mouse breast cancer cells. 66CL4 mouse breast cancer cells were treated with the designated doses of GCT.BC.01. Proliferation was determined using the Agilent Biotek Biospa system and Biospa software.

### Example 4: Glioblastoma

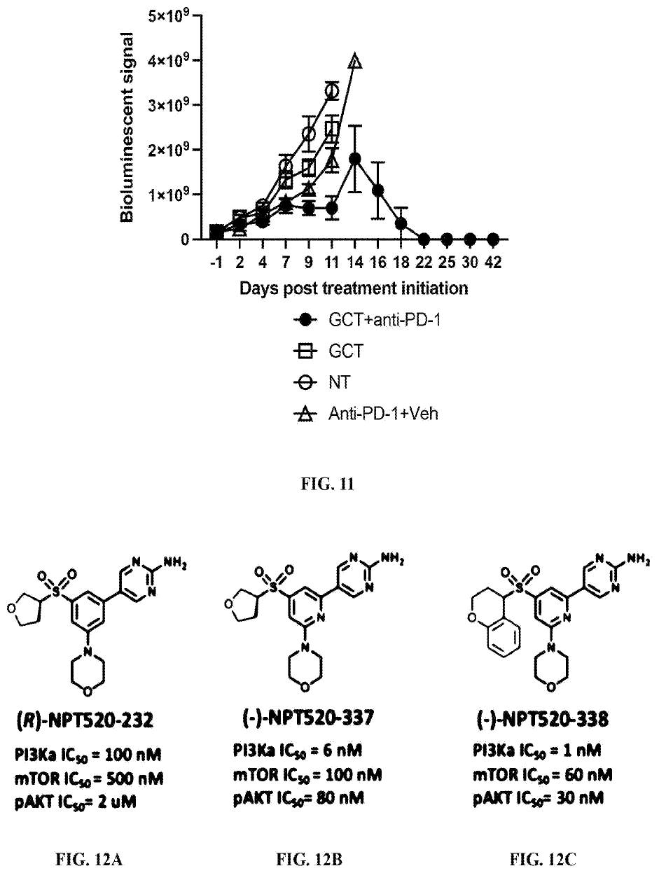

This example refers to FIG. 10 through FIG. 11.

These examples relate to in vivo animal data. These experiments were designed to test the efficacy of GCT.Glio.1 (also known as GCT007, GCT.GLIO.1, NPT520-337) in animal models of GBM and to determine the impact of combining GCT.Glio.1 with immune checkpoint inhibitors by measuring tumor growth in vivo using luciferase tagged tumor and bioluminescence. It was found that the combination of GCT.Glio.1 and anti-PD-1 significantly improved overall survival (FIG. 10) and delayed tumor growth (FIG. 11) compared to either anti-PD-1 or GCT.Glio.1 alone.

FIG. 10, Luciferase flagged GL261 tumor cells were implanted into the caudate nucleus of C57BL6 mice at day 0. Treatments began on day 9. Treatments included no treatment, GCT alone orally administered, anti-PD-1 plus DMSO vehicle, or GCT orally administered followed 24 hours later with the checkpoint inhibitor anti-PD1 as indicated. Tumor growth was confirmed by MRI. Kaplan Meier Survival Curve. The black line indicates animal survival for animals implanted with GL261; the red line indicates survival of animals treated with GCT.Glio.1; The blue line indicates animals treated with GCT.Glio.1 followed 24 hours with treatment with anti-PD1, with the cycle repeating. As of day 28, 3 animals in the combination group are alive.

FIG. 11, Luciferase flagged GL261 tumor cells were implanted into the caudate nucleus of C57B16 mice at day 0. Treatments began on day 9. Treatments included no treatment, GCT alone orally administered, anti-PD-1 plus DMSO vehicle, or GCT orally administered followed 24 hours later with the checkpoint inhibitor anti-PD1 as indicated. The GL261 tumors were measured using bioluminescence and monitored daily. In the upper panel, bioluminescence quantification indicates a significant delay in tumor growth when animals were treated with GCT.Glio.1 followed by the checkpoint inhibitor anti-PD-1.

### Example 5: General Formulations

This example refers to formulations that can be used in the Examples.

FIG. 12. Structures for various compounds referred to in the Examples. (R)-NPT520-232 refers to GCT.2 (GCT.Glio.2), (−)-NPT520-337 refers to GCT.1 (GCT.Glio.1), and (−)-NPT520-338 refers to GCT.Glio.3, and GCT.BR.1.

### In Vitro and In Vivo Formulation Notes for NPT 520-337 Enantiomer 2

### 1. N81520-337: 10 mM Stock in DMSO for In Vitro Use

Weigh out compound. Using the compound's molecular weight, calculate DMSO volume to add to make a 10 mM stock in DMSO. Add this volume and vortex to mix. Store 10 mM stock at −20 C or −80 C and thaw to use. Compound stock in DMSO is stable with repeat freeze thaw. Compound has been confirmed to be soluble up to 10 uM in aqueous buffer/medium.

2. BPT520-337: 10 mg/kg & Vehicle (PO Administration) In Vivo Study

Composition of excipients: 7, 5×NMP, 7.5%-solutol HS-15, 30% PEG-400, 55*(0.95 NaCl) Saline.

### 3. Preparing GCT.1

Add the required volume of NMP (by using pipette) to vial with compound and heat the solution to 65-70 C until the drug is completely dissolved in NMP and then vortex for 30 seconds.

Add required volume of PEG-400 (by using, pipette) and heat for 2 minutes, followed by vortexing the solution. The solution should be clear without any precipitation, cloudiness, or turbidity. Add half of the required volume of saline (via pipette) and vortex the solution for 30 s, do not heat.

Add required volume of Solutol-Hs and vortex for 30 seconds. Flush tip with drug solution twice and make sure Solutol is completely in solution. Vortex. Add the remainder of the saline and vortex for 30 seconds.

### 4. NPYS20-337 (82) Vehicle Preparation:

a. Prepare vehicle by adding NMP, PEG, Solutol Hs, and saline, adding each component one at a time, vortexing for. 30 seconds after each addition.
b. The vehicle is transparent

### Notes:

1. Solutol-Hs is an opaque paste. In a scintiallation vial, heat this paste at 50-60° C. until it becomes liquid.
2. At 10 mg/mL, the suspension in turbid/cloudy. MW=391.44
   IM-391.44 g/L
   10 mM. 3.9144 mg/mL
   21.39
   3,914H1 m9
   21.3 mg

### Example 6: Triple Negative Breast Cancer

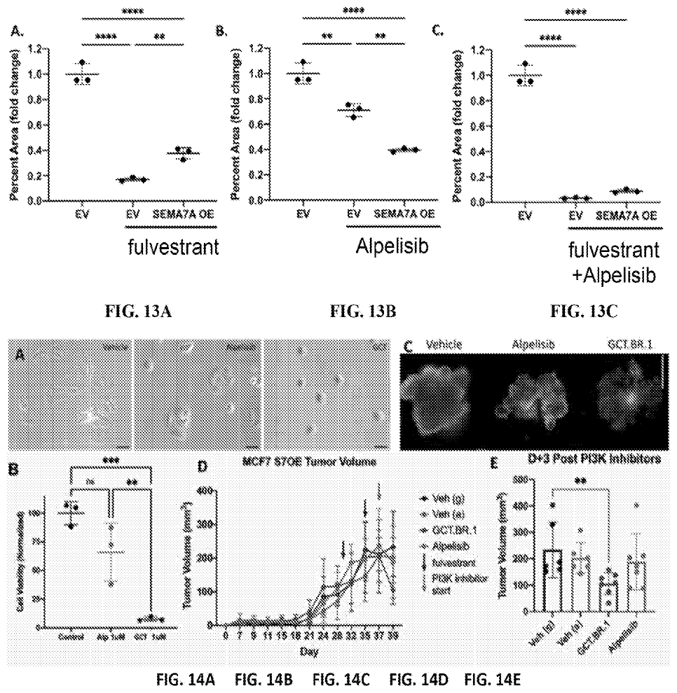

This example refers to FIG. 13 through FIG. 18.

This example refers to endocrine receptor positive cells develop resistance to endocrine therapy via semaphorin 7A and activation of PI3K.

Cell Culture Protocol: Cells were plated in 10 cm plates at ˜1 million cells/plate and incubated overnight to allow adherence. Cell lines and media that were used included McCoy's 5a modified media for HER2+ breast cancer cell line SKBR3 cells, and for MDA-MB-231, MDA-MB-468, MCF-7, and HS578t, culture medium was DMEM. Cells were then treated with the PI3K inhibitor: 0 uM (control), 1 uM, or 10 uM. Treated cells incubated for ˜72 hours. After incubation, cells were imaged, then harvested with 0.25% trypsin/EDTA and counted using trypan blue viability stain with Nexcelom Cellometer Mini. The MDA-MB-231 cell line is an epithelial, human breast cancer cell line that was established from a pleural effusion of a patient with metastatic mammary adenocarcinoma.

The purpose of the following studies was to determine if GCT007 (also described as NPT520-337. NPT520-337/ENT2, or GCT.Br.1), as a potent PI3K inhibitor, can reverse semaphorin 7A-dependent resistance to endocrine therapy for breast cancer. SEMA7A is a membrane-bound protein that can inhibit tumor cell death via integrin-mediated activation of PI3K/Akt pro-survival signaling. The Traci Lyons lab have shown that Semaphorin 7a (SEMA7A) is a potential biomarker for endocrine therapy resistance, increased relapse and decreased overall survival in ER+ BC patients. They found that SEMA7A promotes tumor growth, angio- and lymphangiogenesis, epithelial-to-mesenchymal transition (EMT), metastasis and endocrine therapy resistance in pre-clinical models. Specifically, they have shown that SEMA7A+ ER+ MCF7 tumors result in lung metastases that are resistant to the endocrine therapy, fulvestrant, in part, via downregulation of ER in vivo—posing the need to identify novel, druggable targets for SEMA7A+ ER+ BC. Thus, the following data support the use of GCT007 as a novel therapy for endocrine resistant breast cancer. See, for example, FIG. 13 and FIG. 14.

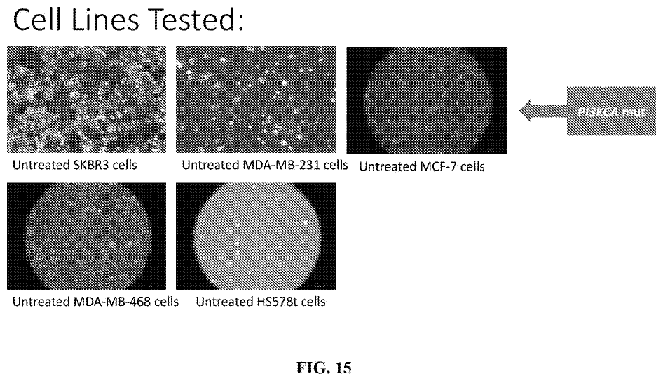

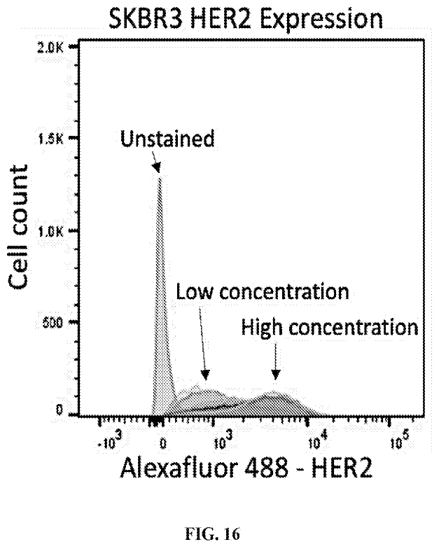

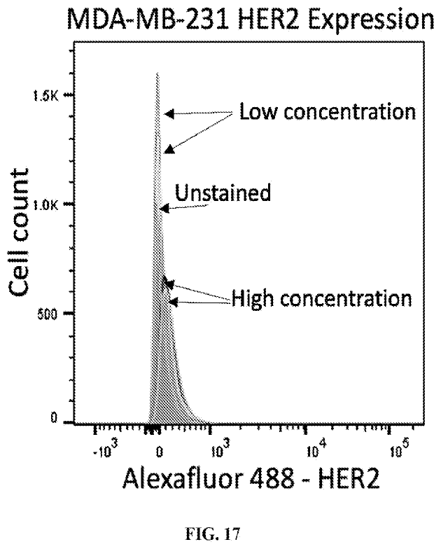

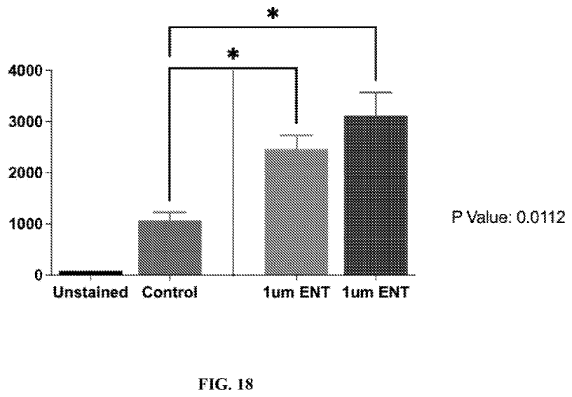

The purpose of the following studies was to test the hypothesis that treatment with GCT007 (also described as NPT520-337/ENT2, or GCT.Br.1) increases cell surface expression of important cell surface receptors, including human epidermal growth factor 2 (HER2) HER2 is an important receptor that provides a required drug target for treatment with the breast cancer drug Herceptin™. Thus, treatment with GCT007 could sensitize HER2 negative and triple negative breast cancer cells to Herceptin™, thereby, providing a novel therapeutic approach of combining GCT007 with the drug Herceptin™, for the treatment of breast cancer. See, for example, FIG. 15, FIG. 16, FIG. 17, and FIG. 18.

FIG. 13A, FIG. 13B, and FIG. 13C. SEMA7A over-expressing (OE) cells respond poorly to endocrine therapy but become sensitive with the addition of PI3K inhibition. MCF7EV and SEMA7A OE cells were treated for 48 hours with A) fulvestrant (7.5 nM), B} Alpelisib (1 uM), or C) the combination.

FIG. 14A, FIG. 14B, FIG. 14C, FIG. 14D, and FIG. 14E. SEMA7A-expressing cells are sensitive to both Alpelisib and a novel PI3K inhibitor (GCT.BR.1) FIG. 14A) MCF7 cells treated with vehicle, 1 uM Alpelisib and 1 uM GCT.BR.1. Scalebar=10 microns. FIG. 14B) Quantification of A. FIG. 14C) MCF7 S7 OE spheroids in suspension were treated with vehicle (DMSO), 1 uM Alpelisib or 0.5 uM GCT and stained with Calcein green viability dye after 48 h. FIG. 14D) Tumor volume in MCF7 SEMA7 A OE-bearing NCG mice, treated with fulvestrant starting on day 30, every 5 days (arrows). FIG. 14E) size matched graphs.

FIG. 15. SK-BR-3 is a human breast cancer cell line that overexpresses the Her2 (Neu/ErbB-2) gene product. These cells display an epithelial morphology in tissue culture and are capable of forming poorly differentiated tumors in immunocompromised mice. MDA-MB-231 is a highly aggressive, invasive and poorly differentiated triple-negative breast cancer (TNBC) cell line as it lacks estrogen receptor (ER) and progesterone receptor (PR) expression, as well as HER2 (human epidermal growth factor receptor 2) amplification.

FIG. 16. SKBR3 cells treated with the labeled doses of GCT.BR.1. Histogram depicting the changes in expression of HER2 analyzed by flow cytometry. Treatment with GCT007 caused a dose-dependent increase in expression of HER 2 on triple negative SKRB3 breast cancer cells.

FIG. 17. Triple negative breast cancer cell line MDA MB 231 cells treated with the labeled doses of GCT.BR.1 (GCT007). Histogram depicting the changes in expression of HER2 analyzed by flow cytometry. Treatment with GCT007 caused a dose-dependent increase in expression of HER 2 on breast cancer 231 cells

FIG. 18. Quantification of the median fluorescence intensity of HER-2 expression on 231 cells.

The present invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising.” or “having,” “containing,” “involving,” and variations thereof herein, is meant to encompass the items listed thereafter and equivalents thereof as well as additional items. The terms “comprising,” “consisting essentially of” and “consisting of,” notably when in the claims, refers to the legal meaning of those terms as set forth, for example, in the USTPO Manual of Patent Examining Procedure (MPEP), such as section 2111.03. Briefly, the transitional term “comprising”, which is synonymous with “including,” “containing,” or “characterized by,” is inclusive or open-ended and does not exclude additional, unrecited elements or method steps: the transitional phrase “consisting of” excludes any element, step, or ingredient not specified in the claim and refers to closing the claim to the inclusion of materials other than those recited except for impurities ordinarily associated therewith; and the transitional phrase “consisting essentially of” limits the scope of a claim to the specified materials or steps and those that do not materially affect the basic and novel characteristic(s) of the claimed invention.

All publications, including patent documents and scientific articles, referred to in this application and the bibliography and attachments are incorporated by reference in their entirety for all purposes to the same extent as if each individual publication were individually incorporated by reference.

All headings and titles are for the convenience of the reader and should not be used to limit the meaning of the text that follows the heading, unless so specified.

What is claimed is:

1. A method of treating a subject with cancer, comprising:
a) providing a subject in need of treatment for at least one cancer;
wherein said cancer is glioblastoma, breast cancer, triple negative breast cancer, or a combination thereof,
b) providing at least one pharmaceutical composition comprising:
1) at least one PI3K inhibitor in a pharmaceutically effective amount in a pharmaceutically acceptable carrier; and
2) at least one checkpoint inhibitor in a pharmaceutically effective amount in a pharmaceutically acceptable carrier;
c) administering a pharmaceutically effective amount of said at least one pharmaceutical composition to said subject;
wherein said subject is treated for said at least one cancer.

2. The method of claim 1,
further comprising administering at least one treatment to said subject.

3. The method of claim 1,
wherein said treatment comprises at least one chemotherapy, at least one immunotherapy, at least one radiation, at least one low temperature, at least one high temperature, at least one laser, at least one surgery, at least one nanoparticle delivery system, or a combination thereof.

4. The method of claim 1,
wherein said at least one PI3K inhibitor and said checkpoint inhibitor are provided together or separately.

5. The method of claim 1,
wherein said at least one PI3K inhibitor comprises: 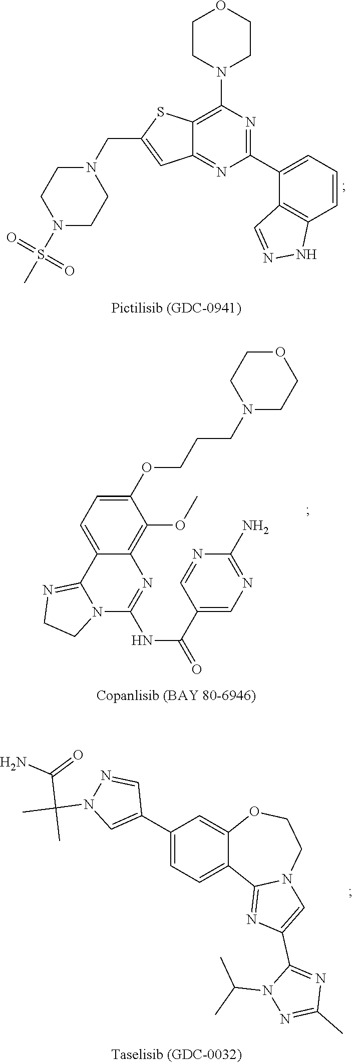 [SMILES: CS(=O)(=O)N1CCN(Cc2cc3nc(-c4cccc5[nH]ncc45)nc(N4CCOCC4)c3s2)CC1 ; COc1c(OCCCN2CCOCC2)ccc2c1N=C(NC(=O)c1cnc(N)nc1)N1CCN=C21 ; Cc1nc(-c2cn3c(n2)-c2ccc(-c4cnn(C(C)(C)C(N)=O)c4)cc2OCC3)n(C(C)C)n1] 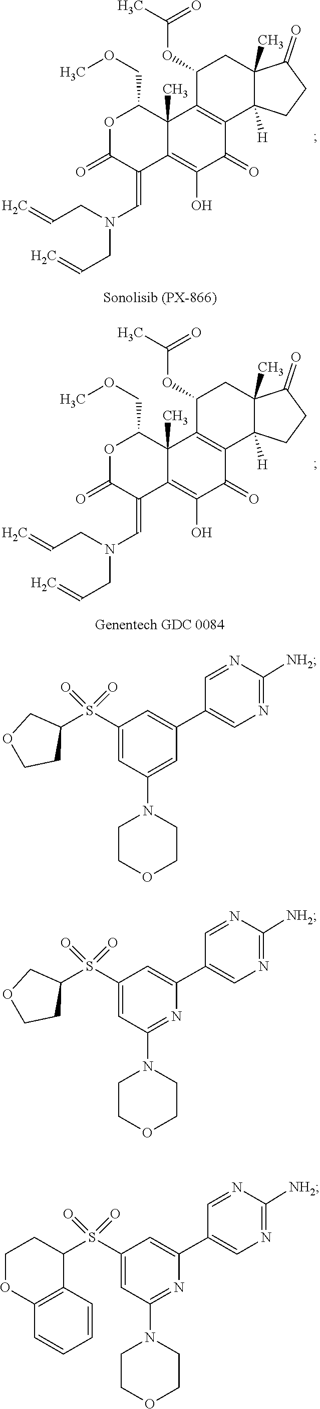 [SMILES: C=CCN(/C=C1\C(=O)O[C@H](COC)[C@@]2(C)C1=C(O)C(=O)C1=C2[C@H](OC(C)=O)C[C@]2(C)C(=O)CC[C@@H]12)CC=C ; C=CCN(/C=C1\C(=O)O[C@H](COC)[C@@]2(C)C1=C(O)C(=O)C1=C2[C@H](OC(C)=O)C[C@]2(C)C(=O)CC[C@@H]12)CC=C ; Nc1ncc(-c2cc(N3CCOCC3)cc(S(=O)(=O)[C@H]3CCOC3)c2)cn1 ; Nc1ncc(-c2cc(S(=O)(=O)[C@H]3CCOC3)cc(N3CCOCC3)n2)cn1 ; Nc1ncc(-c2cc(S(=O)(=O)C3CCOc4ccccc43)cc(N3CCOCC3)n2)cn1]
or a combination thereof.

6. The method of claim 1,
wherein said at least one PI3K inhibitor comprises: 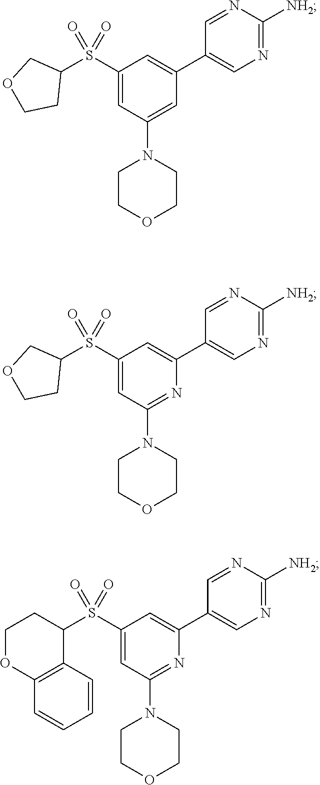 [SMILES: Nc1ncc(-c2cc(N3CCOCC3)cc(S(=O)(=O)C3CCOC3)c2)cn1 ; Nc1ncc(-c2cc(S(=O)(=O)C3CCOC3)cc(N3CCOCC3)n2)cn1 ; Nc1ncc(-c2cc(S(=O)(=O)C3CCOc4ccccc43)cc(N3CCOCC3)n2)cn1]
or a combination thereof.

7. The method of claim 1,
wherein said at least one checkpoint inhibitor comprises:
a) at least one anti-CTLA-4 inhibitor;
b) at least one PD-1 inhibitor;
c) at least one anti-PD-L1 inhibitor; or
d) a combination thereof.

8. The method of claim 7,
wherein said anti-CTLA-4 inhibitor comprises:
ipilimumab.

9. The method of claim 7,
wherein said PD-1 inhibitor comprises:
pembrolizumab, nivolumab, cemiplimab (trade name LIBTAYO®), and dostarlimab (trade name JEMPERLI®), or a combination thereof.

10. The method of claim 7,
wherein said anti-PD-L1 inhibitor comprises:
atezolizumab (trade name TECENTRIQ®), durvalumab (trade name IMFINZI®), and Avelumab (trade name BAVENCIO®), or a combination thereof.

11. A pharmaceutical composition, comprising:
a) at least one PI3K inhibitor in a pharmaceutically effective amount in a pharmaceutically acceptable carrier; and
b) at least one checkpoint inhibitor in a pharmaceutically effective amount in a pharmaceutically acceptable carrier.

12. The pharmaceutical composition of claim 11,
wherein said at least one PI3K inhibitor and said checkpoint inhibitor are provided together or separately.

13. The pharmaceutical composition of claim 11,
wherein said at least one PI3K inhibitor comprises: 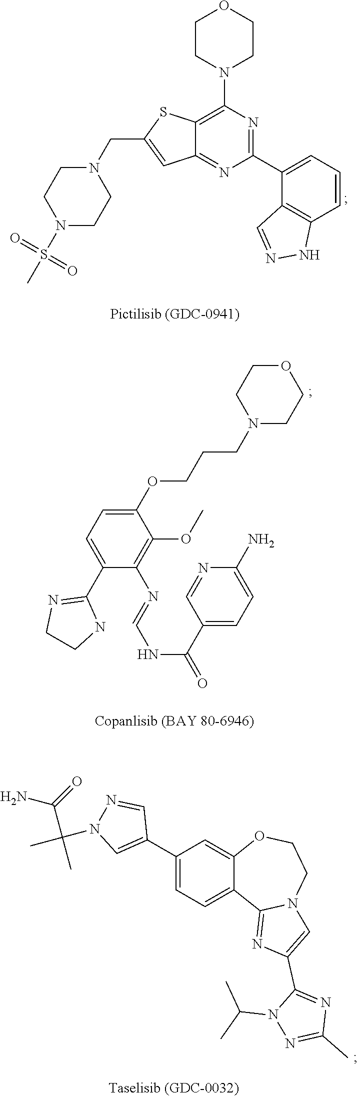 [SMILES: CS(=O)(=O)N1CCN(Cc2cc3nc(-c4cccc5[nH]ncc45)nc(N4CCOCC4)c3s2)CC1 ; COc1c(OCCCN2CCOCC2)ccc(C2=NCCN2)c1/N=C/NC(=O)c1ccc(N)nc1 ; Cc1nc(-c2cn3c(n2)-c2ccc(-c4cnn(C(C)(C)C(N)=O)c4)cc2OCC3)n(C(C)C)n1] 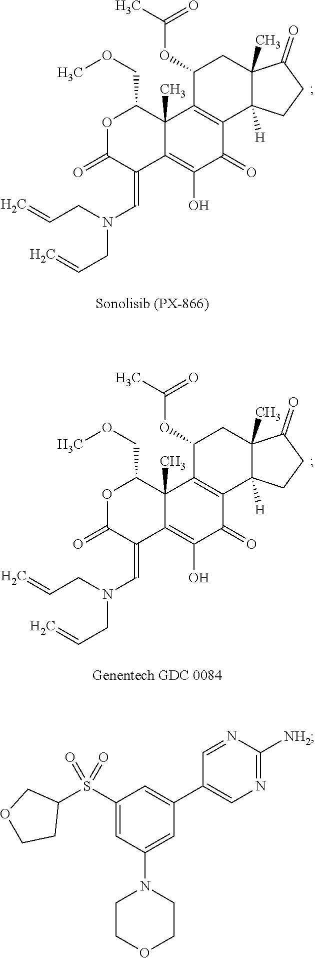 [SMILES: C=CCN(/C=C1\C(=O)O[C@H](COC)[C@@]2(C)C1=C(O)C(=O)C1=C2[C@H](OC(C)=O)C[C@]2(C)C(=O)CC[C@@H]12)CC=C ; C=CCN(/C=C1\C(=O)O[C@H](COC)[C@@]2(C)C1=C(O)C(=O)C1=C2[C@H](OC(C)=O)C[C@]2(C)C(=O)CC[C@@H]12)CC=C ; Nc1ncc(-c2cc(N3CCOCC3)cc(S(=O)(=O)C3CCOC3)c2)cn1] 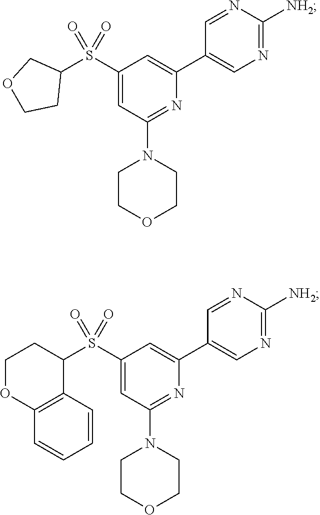 [SMILES: Nc1ncc(-c2cc(S(=O)(=O)C3CCOC3)cc(N3CCOCC3)n2)cn1 ; Nc1ncc(-c2cc(S(=O)(=O)C3CCOc4ccccc43)cc(N3CCOCC3)n2)cn1]
or a combination thereof.

14. The pharmaceutical composition of claim 11,
wherein said at least one PI3K inhibitor comprises: 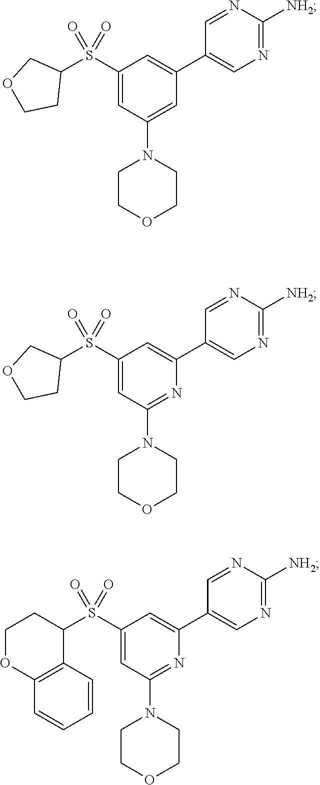 [SMILES: Nc1ncc(-c2cc(N3CCOCC3)cc(S(=O)(=O)C3CCOC3)c2)cn1 ; Nc1ncc(-c2cc(S(=O)(=O)C3CCOC3)cc(N3CCOCC3)n2)cn1 ; Nc1ncc(-c2cc(S(=O)(=O)C3CCOc4ccccc43)cc(N3CCOCC3)n2)cn1]
or a combination thereof.

15. The pharmaceutical composition of claim 11,
wherein said at least one checkpoint inhibitor comprises:
a) at least one anti-CTLA-4 inhibitor;
b) at least one PD-1 inhibitor;
c) at least one anti-PD-L1 inhibitor; or
d) a combination thereof.

16. The pharmaceutical composition of claim 15,
wherein said anti-CTLA-4 inhibitor comprises:
ipilimumab.

17. The pharmaceutical composition of claim 15,
wherein said PD-1 inhibitor comprises:
pembrolizumab, nivolumab, cemiplimab (trade name LIBTAYO®), and dostarlimab (trade name JEMPERLI®), or a combination thereof.

18. The pharmaceutical composition of claim 15,
wherein said anti-PD-L1 inhibitor comprises:
atezolizumab (trade name TECENTRIQ®), durvalumab (trade name IMFINZI®), and Avelumab (trade name BAVENCIO®), or a combination thereof.

19. A method of treating a subject with cancer, comprising:
a) providing a subject in need of treatment for at least one cancer;
b) providing at least one pharmaceutical composition of claim 11;
c) administering a pharmaceutically effective amount of said at least one pharmaceutical composition to said subject;
wherein said subject is treated for said at least one cancer.

20. The method of claim 19,
further comprising administering at least one treatment to said subject.

21. The method of claim 20,
wherein said treatment comprises at least one chemotherapy, at least one immunotherapy, at least one radiation, at least one low temperature, at least one high temperature, at least one laser, at least one surgery, at least one nanoparticle delivery system, or a combination thereof.

22. A method of screening at least one test compound, at least one procedure, or combinations thereof for their ability to make at least one cancer cell more susceptible to one or more treatments for the reduction of cancer cell numbers in a population of cells, comprising:
a. providing one or more cancer cells that expresses one or more markers;
b. providing one or more of said at least one test compound, at least one procedure, or combinations thereof;
c. contacting said one or more cancer cells with said one or more test compounds;
d. measuring the change in expression of said one or more markers by said one or more cancer cells in the presence or absence of said one or more test compounds;
wherein a change in expression of said one or more markers in said one or more cancer cells in the presence of said one or more test compounds relative to in the absence of said one or more test compounds is reasonably predictive of said one or more cancer cells being more susceptible to said one or more treatments for the reduction of cancer cell numbers.

23. The method of claim 22,
wherein said test compounds, procedures, or combinations thereof comprise at least one chemical compound, at least one chemotherapy, at least one immunotherapy, at least one radiation, at least one low temperature, at least one high temperature, at least one laser, at least one surgery, at least one nanoparticle delivery system, or a combination thereof.

24. The method of claim 23,
wherein said nanoparticle delivery system comprises at least one scintillator.

25. The method of claim 23,
wherein said nanoparticle delivery system comprises at least one cleavable linker.

26. The method of claim 25,
wherein said cleavable linker is between the drug and the nanoparticle.

27. The method of claim 22,
wherein said cancer cells are derived from endoderm, mesoderm, ectoderm, or a combination thereof.

28. The method of claim 22,
wherein said cancer cells comprise carcinoma cells, sarcoma cells, leukemia cells, lymphoma cells, myeloma cells, or a combination thereof.

29. The method of claim 22,
wherein said cancer cells comprise skin cells, melanoma cells, non-melanoma cells, brain cells, prostate cells, breast cells, colorectal cells, kidney cells, bladder cells, non-Hodgkin's lymphoma cells, thyroid cells, endometrial cells, head cells, neck cells, brain cells, glioblastoma cells, or a combination thereof.

30. The method of claim 22,
wherein said more susceptible to treatments comprises said cancer cells being more susceptible to cell death, reduction in growth rate, increase in doubling time, more susceptible to DNA damage, less able to repair DNA damage, more likely to be recognized by an immune cell, or a combination thereof.

31. The method of claim 22,
wherein said treatments for reduction of cancer cell numbers comprises at least one chemotherapy, at least one immunotherapy, at least one radiation, at least one low temperature, at least one high temperature, at least one laser, at least one surgery, at least one nanoparticle delivery system, or a combination thereof.

32. The method of claim 22,
wherein said populations of cells comprises a pure culture, a mixed culture, a biopsy, in vivo, in situ, or a combination thereof.

33. The method of claim 22,
wherein said one or more markers comprise one or more of:
i. PD-L1;
ii. MHC Class II;
iii. CD80;
iv. CD86;
v. CD40;
vi. CD95;
vii. CD40
viii. MHC Class I
ix. Lag-3
x. Vista
xi. LC3;
xii. one or more of: CD95, CD44, CD47, TRA-1-60, SSEA-1, EpCam, ALDH1A1, Lgr5, CD13, CD19, CD20, CD24, CD26, CD27, CD34, CD38, CD44, CD45, CD47, CD49f, CD66c, CD90, CD166, TNFRSF16, CD105, CD133, CD117/c-kit, CD138, CD151, CD166, and combinations thereof; and
xiii. combinations thereof.

34. The method of claim 22,
wherein said measuring change in expression comprises immunological methods, nucleic acid methods, proteomic methods, flow cytometric methods, genomic methods, or a combination thereof a pure culture, a mixed culture, a biopsy, or a combination thereof.

35. The method of claim 22,
wherein said change in expression comprises an increase, decrease, or combination thereof from a pure culture, a mixed culture, a biopsy, or a combination thereof.

36. The method of claim 22,
wherein said reasonably predictive comprises being a statistically significant difference at a p value of less than 0.10, 0.05, or as acceptable to those in the art.

37. The method of claim 22;
further comprising testing said cancer cells with said one or more test compounds with said one or more treatments for the reduction of cancer cell numbers.

38. A compound identified by the method of claim 22.

39. A pharmaceutical composition, comprising:
the compound of claim 38 in a pharmaceutically effective amount in a pharmaceutically acceptable carrier.

40. The pharmaceutical composition of claim 39,
further comprising at least one additional cancer treatment.

41. The pharmaceutical composition of claim 40,
wherein said treatment comprises at least one chemotherapy, at least one immunotherapy, at least one radiation, at least one low temperature, at least one high temperature, at least one laser, at least one surgery, at least one nanoparticle delivery system, or a combination thereof.

42. A method of treating a subject with cancer, comprising:
a. providing a subject in need of treatment for at least one cancer;
b. providing at least one compound or pharmaceutical composition of claim 38;
c. administering a pharmaceutically effective amount of said at least one compound to said subject;
wherein said subject is treated for said at least one cancer.

43. The method of claim 42,
further comprising administering at least one treatment to said subject.

44. The method of claim 43,
wherein said treatment comprises at least one chemotherapy, at least one immunotherapy, at least one radiation, at least one low temperature, at least one high temperature, at least one laser, at least one surgery; at least one nanoparticle delivery system, or a combination thereof.
